(19) United States
(12) Patent Application Publication
(10) Pub. No.: US 2016/0000946 A1
(43) Pub. Date: Jan. 7, 2016

(54) RADIOLABELED ANTI-GLYPICAN-3 IMMUNOCONJUGATES FOR IMMUNO-PET IMAGING OF HEPATOCELLULAR CARCINOMA

(71) Applicant: THE BOARD OF TRUSTEES OF THE LELAND STANFORD JUNIOR UNIVERSITY, Palo Alto, CA (US)

(72) Inventors: ZHEN CHENG, Mountain View, CA (US); MEI-SZE CHUA, Menio Park, CA (US); SAMUEL SO, Atherton, CA (US); XIAOYANG YANG, South San Francisco, CA (US)

(21) Appl. No.: 14/773,162

(22) PCT Filed: Mar. 7, 2014
(86) PCT No.: PCT/US14/21898

§ 371 Date: Sep. 4, 2015

Related U.S. Application Data
(60) Provisional application No. 61/781,172, filed on Mar. 14, 2013.

(51) Int. Cl. A61K 51/10; G01N 33/534; G01N 33/574; C07K 16/30

(52) U.S. Cl. CPC A61K 51/1057; C07K 16/303; G01N 33/534; G01N 33/57438; C07K 2317/40; G01N 2333/4722

(57) ABSTRACT

Provided are immunoconjugate probes useful for detecting hepatocellular carcinoma (HCC) lesions. The probes comprise a glypican-3 (GPC3)-specific monoclonal antibody or fragment thereof conjugated to a radionuclide such as ⁸⁹Zr, ⁶⁴Cu, and the like. The probes are useful for obtaining PET images with high tumor-to-liver ratios and targeting for diagnostic imaging of HCC lesions or cells in vitro and in vivo.

### CROSS-REFERENCE TO RELATED APPLICATIONS

This application claims priority to U.S. Provisional Patent Application Ser. No. 61/781,172 entitled “RADIOLABELED ANTI-GLYPICAN-3 MONOCLONAL ANTIBODY FOR IMMUNO-PET IMAGING OF HEPATOCELLULAR CARCINOMA” filed on Mar. 14, 2013, the entirety of which is hereby incorporated by reference.

### TECHNICAL FIELD

The present disclosure is generally related to glypican-3-specific probes suitable for use in PET imaging of hepatocellular carcinoma cells. The present disclosure is further related to methods of using said probe compositions to image and detect hepatocellular carcinomas and cells thereof, both in vivo and in vitro.

### BACKGROUND

Hepatocellular carcinoma (HCC) is the fifth most prevalent malignancy and the third leading cause of cancer-related deaths worldwide (Befeler & Di Bisceglie (2002) Gastroenterology 122: 1609-1619; Okuda K. (2000) J. Hepatol. 32: 225-237; Schafer & Sorrell (1999) Lancet 353:1253-1257). It is typically asymptomatic at the early stage, with only 10-20% of HCC patients being diagnosed early enough for appropriate surgical treatment (Capurro et al., (2003) Gastroenterology 125: 89-97). The delayed diagnosis of HCC is associated with limited treatment options and much lower chances of survival (Bruix & Llovet (2009) Lancet 373: 614-616; Bruix & (2005) Hepatology 42: 1208-1236; Massarweh et al., (2010) J. Am. Coll. Surg. 210: 441-448). Therefore, the early and accurate detection of HCC is fundamental to improving its currently dismal prognosis. Current diagnostic imaging of HCC is heavily reliant on radiological technologies such as ultrasonography, computed tomography (CT), and magnetic resonance imaging (MRI) (Murakami et al., (1995) Acta. Radiol. 36: 372-376). However, these traditional technologies are often unable to detect HCC lesions of less than about 2 cm, and are incapable of differentiating between HCC lesions and other benign liver lesions such as cirrhotic and dysplastic nodules, leading to false-positive diagnoses (Coston et al., (2008) Am. J. Surg. Pathol. 32: 433-444). Considering these limitations and the increasing global incidence and mortality of HCC, more accurate and sensitive diagnostic approaches are urgently needed.

Molecular imaging of cancer based on imaging of specific molecular targets associated with cancer has the potential to enable early diagnosis and clinical management of cancer patients (Gambhir S.S. (2002) Nat. Rev. Cancer 2: 683-693; Weissleder R. (2002) Nat. Rev. Cancer 2: 11-18). The selection of a molecular target that is specifically over-expressed in the tumor of interest is critical to the success of molecular imaging. In HCC, the heparin sulfate proteoglycan glypican-3 (GPC3) is a potentially valuable molecular target for diagnostic imaging for several reasons: (i) GPC3 is a 60 kDa cell-surface protein that is attached to the cell membrane via a glycosylphosphatidylinositol anchor (Filmus J. (2001) Glycobiology 11: 19R-23R), making it readily accessible for antibody-mediated targeting and binding; (ii) GPC3 is expressed in a high percentage of HCC patients (Capurro et al., (2003) Gastroenterology 125: 89-97; Hsu et al., (1997) Cancer Res. 57: 5179-5184; Nakatsura et al., (2003) Biochem. Biophys. Res. Commun. 306: 16-25). Specifically, GPC3 was reported to be expressed in 53-78% of well-differentiated, 86-93% of moderately differentiated, and 86-100% of poorly differentiated HCCs (Kandil & Cooper (2009) Adv. Anat. Pathol. 16: 125-129; Wang et al., (2006) Hum. Pathol. 37: 1435-1441; Yamauchi et al., (2005) Mod. Pathol. 18: 1591-1598); and (iii) GPC3 has the ability to distinguish pre-neoplastic and benign liver lesions from malignant HCCs (Coston et al., (2008) Am. J. Surg. Pathol. 32: 433-444; Nakatsura et al., (2003) Biochem. Biophys. Res. Commun. 306: 16-25; Wang et al., (2006) Hum. Pathol 37: 1435-1441; Shirakawa et al., (2009) Int. J. Oncol. 34: 649-656).

While over-expressed in HCC, normal livers and tissues adjacent to HCCs have negligible expression of GPC3 (Luo et al., (2006) Hepatology 44: 1012-1024). Importantly, GPC3 expression was found to be consistently much higher in small HCCs than in cirrhosis and other types of small focal lesions, suggesting that the transition from premalignant lesions to small HCC is usually associated with a sharp increase in GPC3 expression (Capurro et al., (2003) Gastroenterology 125: 89-97; Nakatsura et al., (2003) Biochem. Biophys. Res. Commun. 306: 16-25; Wang et al., (2006) Hum. Pathol 37: 1435-1441; Di Tommaso et al., (2007) Hepatology 45: 725-734; Wang et al., (2008) Arch. Pathol. Lab. Med. 132: 1723-1728). Taken together, GPC3 is one of the earliest proteins expressed as a hepatocyte transforms into the malignant phenotype and is, therefore, a potentially useful molecular target for the early detection of HCC.

Molecular imaging with positron emission tomography (PET) using tumor-seeking radiolabeled-molecules has gained wide acceptance in oncology, allowing earlier diagnosis and better clinical management of cancer patients (Gambhir S. S. (2002) Nat. Rev. Cancer 2: 683-693; Weissleder R. (2002) Nat. Rev. Cancer 2: 11-18; Fletcher et al., (2008) J. Nucl. Med. 49: 480-508). A variety of molecules, including glucose analogues, monoclonal antibodies (mAbs), antibody fragments, and peptides, can be used as tumor-seeking molecules with different levels of tumor accessibility and specificity. Among them, despite their large molecular size and pharmacokinetic limitations, monoclonal antibodies represent the best candidates with highest specificity in tumor detection, and have been widely used in many clinical applications. For example, it has been documented that a monoclonal antibody against human epidermal growth factor receptor 2 (HER2) is being used in the detection and treatment of primary and metastatic breast cancer (Dijkers et al., (2010) Clin. Pharmacol. Ther. 87: 586-592; Piccart-Gebhart et al., (2005) N. Engl. J. Med. 353: 1659-1672; Slamon et al., (2001) N. Engl. J. Med. 344: 783-792). However, the use of monoclonal antibodies for imaging of liver cancer represents a major challenge, as the liver is primarily responsible for the clearance of any exogenous molecule. As such, the use of monoclonal antibody-based PET probes for imaging of liver tumors typically results in high liver uptakes and poor tumor-to-liver ratios. The successful imaging of liver tumors, therefore, requires the combined selection of a highly specific target molecule such as GPC3, as well as effective approaches to decrease non-specific liver uptake.

Early detection of HCC is crucial and may significantly change its outcome. Considering the poor performance of current imaging modalities for HCC diagnosis, there is an continuing need to develop new molecular imaging techniques which can dramatically improve the sensitivity and specificity of HCC detection (Capurro et al., (2003) Gastroenterology 125: 89-97; Coston et al., (2008) Am. J. Surg. Pathol. 32: 433-444).

### SUMMARY

Provided are immunoconjugate probes useful for detecting hepatocellular carcinoma (HCC) lesions. The probes comprise a glypican-3 (GPC3)-specific monoclonal antibody or fragment thereof conjugated to a radionuclide such as ⁸⁹Zr, ⁶⁴Cu, and the like. The probes are useful for obtaining PET images with high tumor-to-liver ratios and targeting for diagnostic imaging of HCC lesions or cells in vitro and in vivo.

One aspect of the disclosure encompasses embodiments of an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT.

In embodiments of this aspect of the disclosure, the detectable label can be a radionuclide selected from the group consisting of: ⁶⁴Cu, ⁶⁷Cu, ⁸⁹Zr, ¹²⁴I, ⁸⁶Y, ⁹⁰Y, ¹¹¹In, _{123/131}I, ¹⁷⁷Lu, ¹⁸F, and ^{99m}Tc.

In embodiments of this aspect of the disclosure, the detectable label can be detectable by positron emission tomography (PET) and is zirconium⁸⁹ (⁸⁹Zr) or copper⁶⁴(⁶⁴Cu).

In embodiments of this aspect of the disclosure, the detectable label can be attached to the anti-GPC3-specific antibody (mAb), or a target-specific fragment thereof, by a linker.

In some embodiments of this aspect of the disclosure, the linker can be DFO.

Another aspect of the disclosure encompasses embodiments of a pharmaceutically acceptable composition comprising: an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT, and further comprising a pharmaceutically acceptable carrier.

Yet another aspect of the disclosure encompasses embodiments of a method of obtaining an image of a hepatocellular carcinoma in a subject animal or human, the method comprising the steps of: (a) delivering to a subject animal or human a pharmaceutically acceptable composition comprising an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT; (b) subjecting the subject animal or human to positron emission tomography; (c) identifying a detectable signal from the probe in the subject animal or human; and (d) generating an image of the detectable signal, thereby obtaining an image of a hepatocellular carcinoma in a subject animal or human.

In embodiments of this aspect of the disclosure, the detectable label can be zirconium⁸⁹ (⁸⁹Zr) or copper⁶⁴ (⁶⁴Cu).

In embodiments of this aspect of the disclosure, the detectable PET label can be attached to the anti-GPC3-specific antibody (mAb) or the target-specific fragment thereof by a linker. In some embodiments of this aspect of the disclosure, the linker can be DFO.

Still another aspect of the disclosure encompasses embodiments of a method of detecting a cell having glypican-3 (GPC3), or population of said cells, in a biological sample, the method comprising the steps of: (a) obtaining a biological sample from an animal or human subject; (b) contacting the biological sample with an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT; and (c) subjecting the biological sample to positron emission tomography, whereupon a detectable signal from the probe indicates the presence of a cell having glypican-3 (GPC3), or population of said cells, in the biological sample.

In embodiments of this aspect of the disclosure, the detectable label can be zirconium⁸⁹ (⁸⁹Zr).

In embodiments of this aspect of the disclosure, the detectable PET label can be attached to the anti-GPC3-specific antibody (mAb) or the target-specific fragment thereof by a linker. In some embodiments of this aspect of the disclosure, the linker can be DFO.

Still another aspect of the disclosure encompasses embodiments of a method of determining if a subject animal or human has a hepatocellular carcinoma expressing glypican-3 (GPC3), the method comprising the steps of: (a) obtaining a biological sample from an animal or human subject; (b) contacting the biological sample with an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT; (c) subjecting the biological sample to positron emission tomography; and (d) identifying a detectable signal from the probe, wherein the detection of the probe indicates the presence of a cell having glypican-3 (GPC3), or population of said cells, in the biological sample, thereby indicating the presence of a hepatocellular carcinoma in the subject animal or human.

In embodiments of this aspect of the disclosure, the detectable PET label can be zirconium⁸⁹ (⁸⁹Zr).

In embodiments of this aspect of the disclosure, the detectable PET label can be attached to the anti-GPC3-specific antibody (mAb) or the target-specific fragment thereof by a linker. In some embodiments of this aspect of the disclosure, the linker can be DFO.

### BRIEF DESCRIPTION OF THE DRAWINGS

Aspects of the present disclosure will be more readily appreciated upon review of the detailed description of its various embodiments, described below, when taken in conjunction with the accompanying drawings. The drawings are described in greater detail in the description and examples below.

FIGS. 1A-1D illustrate that anti-GPC3 mAb binds to recombinant human GPC3, and specifically identifies GPC3-expressing HCC cells.

FIG. 1A is a graph illustrating the binding of an anti-GPC3 mAb to recombinant human GPC3 protein assessed using an affinity binding assay. Fluorescence counts corresponding to each serial dilution of the anti-GPC3 mAb were measured (n=3). The mean K_{D} value was determined to be 0.4507±0.04562 nM.

FIGS. 1B and 1C are digital images illustrating GPC3 protein expression levels measured in various human HCC cell lines and the non-HCC PC3 cell line by Western blot analysis (FIG. 1B) and immunofluorescence staining (FIG. 1C). For immunofluorescence, anti-GPC3 mAb was used as the primary antibody, and ALEXA FLOUR.RTM 660 goat anti-mouse IgG was used as the secondary antibody. Overlay images of GPC3 staining and DAPI stained cell nuclei are shown.

FIG. 1D illustrates digital images showing immunohistochemistry-staining in HepG2 and PLC/PRF/5 xenograft sections.

FIGS. 2A and 2B illustrate specific uptake and cellular internalization of ⁸⁹Zr-DFO-GPC3 mAb.

FIG. 2A illustrates cellular uptake of ⁸⁹Zr-DFO-GPC3 in HepG2, PLC/PRF/5, SNU449, and PC3 cells over time at 37° C.

FIG. 2B illustrates cell-associated radioactivity as a function of time after incubation of HepG2 cells with ⁸⁹Zr-DFO-GPC. All data are presented as means±SD (n=4).

FIGS. 3A-3C illustrate the distribution of free ⁸⁹Zr in normal mice in vivo. Representative decay-corrected coronal (top) and transaxial (bottom) PET images in normal mice are shown. Free ⁸⁹Zr was used as a PET probe and injected through the tail vein. Specific images of the bladder (FIG. 3A) and kidneys (FIG. 3B) at different time points (1 h, 24 h and 48 h) are shown. Sagittal image of spine (FIG. 3C) at 72 h post-injection is also shown. Scale bars (% ID/g) are shown.

FIGS. 4A-4D illustrate tumor delivery of ⁸⁹Zr-DFO-GPC3 in subcutaneous xenografts in vivo.

FIG. 4A illustrates representative decay-corrected coronal (top) and transaxial (bottom) PET images in HepG2, PLC/PRF/5 and PC3-tumor bearing mice at different at different time points after tail vein injection of ⁸⁹Zr-DFO-GPC3. Arrows indicate the location of the tumors. Scale bars (% ID/g) are shown. Time-activity curves of tumor (FIG. 4B), liver (FIG. 4C) and tumor-to-liver ratios (FIG. 4D) derived from multiple-time point small-animal PET images after tail injection of ⁸⁹Zr-DFO-GPC3. ROI quantification from HepG2 xenografts, PLC/PRF/5 xenografts, and PC3 xenografts are shown in each of FIGS. 4B-4D. Data presented are shown as mean±SD % ID/g (n=4).

FIGS. 5A-5F illustrate tumor delivery of ⁸⁹Zr-DFO-GPC3 in orthotopic HCC xenografts in vivo.

FIG. 5A illustrates representative decay-corrected coronal (top), transaxial (middle) and sagittal (bottom) PET/CT images of HepG2 orthotopic mice. As controls, images from normal mice at every time point (24 h, 48 h, 72 h, 120 h and 168 h) are also shown side-by-side. Scale bars (signal density for CT, and % ID/g for PET) are to the right. Representative decay corrected images for PLC/PRF/5 (FIG. 5B) and Hep3B (FIG. 5C) orthotopic mice at a late time point (168 h p.i) are also shown. Time-activity curves of tumor (FIG. 5D), liver (FIG. 5E) and tumor-to-liver ratios (FIG. 5F) derived from multiple-time point small-animal PET images after tail injection of ⁸⁹Zr-DFO-GPC3 are shown. ROI quantification from HepG2 xenografts, PLC/PRF/5 xenografts, and Hep3B xenografts are shown in each of FIGS. 5D-5F. Data presented are shown as mean±SD % ID/g (n=4).

FIG. 6 illustrates that a ⁶⁴Cu-DOTA-GPC3 probe was superior to the more commonly used PET tracer ¹⁸F-FDG in detecting GPC3-positive HCC xenografts.

FIGS. 7A illustrate the specific uptake and cellular internalization of ⁶⁴Cu-DOTA-GPC3 mAb in GPC3-expressing cells. Cellular uptake of ⁶⁴Cu-DOTA-GPC3 mAb in HepG2 cells (high GPC3 expression), PLC/PRF/5 cells (trace GPC3 expression) and SNU449 cells (no GPC3 expression) over time at 4° C. and 37° C. *, p<0.05.

FIG. 7B illustrates cell-associated radioactivity as a function of time after incubation of HepG2 cells with ⁶⁴Cu-DOTA-GPC3 mAb. The radioactivity removed from cells by treatment with 0.2 M glycine buffer, pH 2.0, was considered as membrane-bound fraction, and the rest as internalized fraction. All data are presented as means±SD (n=4).

FIGS. 8A and 8B illustrate tumor delivery of ⁶⁴Cu-DOTA-GPC3 in vivo. Representative decay-corrected coronal (top) and transaxial (bottom) PET images in HepG2 tumor-bearing mice (FIG. 8A) and in PLC/PRF/5 tumor-bearing mice (FIG. 8B) at different time points after tail vein injection of ⁶⁴Cu-DOTA-GPC3. Arrows indicate the location of the tumors. Scale bars (% ID/g) are shown.

FIGS. 9A-9D illustrate time-activity curve of PET quantification of ⁶⁴Cu-DOTA-GPC3 in subcutaneous mice. Time-activity curves of tumor (FIG. 9A), liver (FIG. 9B), and muscle (FIG. 9C) were derived from multiple-time-point small-animal PET images after tail vein injection of ⁶⁴Cu-DOTA-GPC3. FIG. 9D illustrates tumor-to-liver ratios derived from multiple-time-point PET images are shown. ROI quantification from HepG2 xenografts and PLC/PRF/5 xenograft are shown. Data presented are shown as mean±SD % ID/g (n=3).

FIGS. 10A and 10B illustrate tumor delivery of ⁸⁹Zr-DFO-GPC3 in orthotopic models in vivo. Representative decay-corrected coronal (top), transaxial (middle) and sagittal (bottom) PET/CT images of PLC/PRF/5 (FIG. 10A) and Hep3B (FIG. 10B) orthotopic mice from each time points (24 h, 48 h, 72 h, 120 h and 168 h) are shown. Scale bars (signal density for CT, and % ID/g for PET) are shown.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

### DESCRIPTION OF THE DISCLOSURE

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “ includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

### Definitions

In describing and claiming the disclosed subject matter, the following terminology will be used in accordance with the definitions set forth below.

The term “Glypican-3” as used herein also refers to “glypican proteoglycan 3,” “GPC3,” “GTR2-2,” “SGB,” “DGSX,” “SDYS,” “SGBS,” and “SGBS1” and is contemplated to include variants, isoforms and species homologs of human or animal Glypican-3. Accordingly, human antibodies of this disclosure may, in some instances, cross-react with Glypican-3 from species other than human. In certain embodiments, the antibodies may be completely specific for one or more human Glypican-3 proteins and may not exhibit species or other types of non-human cross-reactivity. The complete amino acid sequence of an exemplary human Glypican-3 has Genbank/NCBI accession number NM_{—}004484 (SEQ ID No: 1).

The term “Positron Emission Tomography (PET)” as used herein refers to a nuclear imaging technique used in the medical field to assist in the diagnosis of diseases. PET allows the physician to examine the whole patient at once by producing pictures of many functions of the human body unobtainable by other imaging techniques. In this regard, PET displays images of how the body works (physiology or function) instead of simply how it looks. PET is considered the most sensitive, and exhibits the greatest quantification accuracy of any nuclear medicine imaging instrument available at the present time. Applications requiring this sensitivity and accuracy include those in the fields of oncology, cardiology, and neurology.

In PET, short-lived positron-emitting isotopes, herein referred to as radiopharmaceuticals, are injected into a patient. When these radioactive drugs are administered to a patient, they distribute within the body according to the physiologic pathways associated with their stable counterparts. For example, the radiopharmaceutical ¹⁸F-labeled glucose, known as fluorodeoxyglucose or “FDG ”, can be used to determine where normal glucose would be used in the brain. Other radioactive compounds include, but are not limited to, ¹¹C-labeled acetate, ¹³N-labeled ammonia, or ¹⁵O-labeled water, ⁶⁴Cu²⁺, ⁸⁹Zr⁺, and the like used to study such phenomena as neoplastic transformation or blood flow.

The term “SPECT” as used herein refers to “Single-Photon Emission Computed Tomography which is a nuclear medicine tomographic imaging technique using gamma rays. It is very similar to conventional nuclear medicine planar imaging using a gamma camera and able to provide true 3D information. This information is typically presented as cross-sectional slices through the patient, but can be freely reformatted or manipulated as required. The basic technique requires delivery of a gamma-emitting radioisotope (called radionuclide) into the patient, normally through injection into the bloodstream. On occasion, the radioisotope is a simple soluble dissolved ion, such as a radioisotope of gallium(III), which happens to also have chemical properties that allow it to be concentrated in ways of medical interest for disease detection. Other useful radioactive compounds include, but are not limited to, ¹¹C-labeled acetate, ¹³N-labeled ammonia or ¹⁵O-labeled water, ⁶⁴Cu, ⁸⁹Zr, and the like.

The terms “administering” and “delivering” as used herein refer to methods of delivering a composition of the disclosure to a subject. Such methods are well known to those skilled in the art and include, but are not limited to, oral, nasal, intravenous, intramuscular, intraperitoneal, subcutaneous, intrathecal, intradermal, or topical administration. Compositions of the disclosure may be administered on a continuous or an intermittent basis. Methods for formulating and subsequently administering compositions are well known to those skilled in the art. See, for example, Remington, 2000, The Science and Practice of Pharmacy, 20th Ed., Gennaro & Gennaro, eds., Lippincott, Williams & Wilkins. The dose administered will depend on many factors, including the mode of administration and the formulation.

The terms “organism”, “host”, and “subject” as used herein refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). “Subject” may, therefore, be a cell, a population of cells, a tissue, an organ, or an organism.

The term “pharmaceutically acceptable carrier” as used herein refers to a diluent, adjuvant, excipient, or vehicle with which a probe of the disclosure can be administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil, and the like. When administered to a patient, the probe and pharmaceutically acceptable carriers can be sterile. Water is a useful carrier when the heterodimeric probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of agents, such as a pH buffering agents. The present compositions advantageously may take the form of solutions, emulsion, sustained-release formulations, or any other form suitable for use.

The terms “specific,” “selectively binding,” and “specific binding” as used herein refer to the specific recognition of one molecule, of two different molecules, compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide interactions, and so forth. In the context of the disclosure, the term “specific” refers to the ability of a monoclonal antibody, or a target-binding fragment thereof, to distinguish one antigenic site from another, and in particular to bind to an epitopic site of a glypican-3 and not to a region of another molecule species.

The term “generating an image” as used herein refers to acquiring a detectable signal generated from a probe according to the present disclosure and determining the location of the source in a cell or an animal or human tissue. The acquisition of the detectable signal according to the disclosure is most advantageously by PET. The intensity of the detectable signal may also be quantified.

The term “cell or population of cells” as used herein refers to an isolated cell or plurality of cells excised from a tissue or grown in vitro by tissue culture techniques. In the alternative, a population of cells may also be a plurality of cells in vivo in a tissue of an animal or human host.

The term “contacting a cell or population of cells” as used herein refers to delivering a composition, such as a composition according to the present disclosure with or without a pharmaceutically or physiologically acceptable carrier, to an isolated or cultured cell or population of cells, or administering the probe in a suitable pharmaceutically acceptable carrier to an animal or human subject. Thereupon, it may be systemically delivered to the target and other tissues of the host, or delivered to a localized target area of the host. Administration may be, but is not limited to, intravenous delivery, intraperitoneal delivery, intramuscularly, subcutaneously or by any other method known in the art. One advantageous method is to deliver the composition directly into a blood vessel leading immediately into a target organ or tissue such as the liver, thereby reducing dilution of the probe in the general circulatory system.

The term “derferoxamine” (also known as desferrioxamine B, desferoxamine B, DFO-B, DFOA, DFB or DESFERAL.RTM) as used herein refers to a bacterial siderophore produced by the actinobacteria Streptomyces pilosus and having the chemical name N′-{5-[acetyl(hydroxy)amino]pentyl{-N-[5-({4-[(5-aminopentyl)(hydroxy)amino]-4-oxobutanoyl}amino)pentyl]-N-hydroxysuccinamide.

The term “antibody” as used herein refers to an immunoglobulin protein that specifically binds to, and is thereby defined as complementary, with a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. It is contemplated that most advantageous for the generation of a probe according to the disclosure, and for use in the methods herein disclosed, that the antibody be a monoclonal antibody that selectively and specifically binds to an epitopic region of a glypican-3 molecule. Such monoclonal antibodies are also commercially available and may be selected for conjugation to a PET-detectable label by methods known in the art. Antibodies useful for incorporation into the immunoconjugates of the disclosure may include a complete immunoglobulin or fragment thereof, which immunoglobulins include the various classes and isotypes, such as IgA, IgD, IgE, IgG1, IgG2a, IgG2b and IgG3, IgM, IgY, etc. Fragments thereof may include Fab, Fv and F(ab′)₂, Fab′, scFv, and the like where appropriate so long as binding affinity for a particular molecule is maintained.

The term “target-specific fragment” of an antibody as used herein refers to one or more fragments of an antibody that retain the ability to specifically bind to glypican-3. It has been shown that the antigen-binding function of an antibody can be performed by fragments of a full-length antibody. Accordingly, it is contemplated to be within the scope of the disclosure for the anti-glypican-3-specifc moiety of the probes to be any fragment of an anti-glypican-3 antibody that can specifically bind to a region of a glypican-3 polypeptide.

Examples of binding fragments encompassed within the term “target-specific fragment” of an antibody include (i) an Fab fragment, a monovalent fragment consisting of the V_{L}, V_{H}, C_{L} and C_{H1} domains; (ii) an F(ab′)₂ fragment, a bivalent fragment comprising two Fab fragments linked by a disulfide bridge at the hinge region; (iii) an Fab′ fragment, which is essentially an Fab with part of the hinge region (see, FUNDAMENTAL IMMUNOLOGY (Paul ed., 3rd ed. 1993); (iv) an Fd fragment consisting of the V_{H} and C_{H1,} domains; (v) an Fv fragment consisting of the V_{L} and V_{H} domains of a single arm of an antibody; (vi) a dAb fragment (Ward et al., (1989) Nature 341: 544-546), which consists of a V_{H} domain; and (vii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “target-specific fragment” of an antibody. These antibody fragments are obtained using conventional techniques known to those with skill in the art, and the fragments are screened for utility in the same manner as are intact antibodies.

The term “isolated antibody” as used herein refers to an antibody substantially free of other antibodies having different antigenic specificities (i.e., an isolated antibody that specifically binds glypican-3 is substantially free of antibodies that specifically bind antigens other than glypican-3). An isolated antibody that specifically binds glypican-3 may, however, have cross-reactivity to other antigens, such as glypican-3 molecules from other species.

The terms “monoclonal antibody” or “monoclonal antibody composition” as used herein refer to a preparation of antibody molecules of a single molecular composition. A monoclonal antibody composition displays a single binding specificity and affinity for a particular epitope.

The term “immunoconjugate” as used herein refers to a composition comprising an immunoglobulin specific for glypican-3, and in particular an extracellular region of a glypican-3, or a glypican-3-specific fragment of such an immunoglobulin and a PET-detectable label attached thereto, either directly or via a linker.

As used herein, a “label” or “tag” refers to a molecule that, when attached to an antibody or antigen-binding fragment thereof, provides or enhances a means of detecting the antibody or fragment thereof. Radionuclides may be either therapeutic or diagnostic; diagnostic imaging using such nuclides is also well known. Most advantageous for the compositions of the disclosure is the isotope ⁸⁹Zr (zirconium-89) although it is contemplated that other radionuclides such as, but not limited to, ⁶⁴Cu, ⁶⁷Cu, ⁸⁹Zr, ¹²⁴I, ⁸⁶Y, ⁹⁰Y, ¹¹¹In, ^{123/131}I, ¹⁷⁷Lu, ¹⁸F, ^{99m}Tc, and the like, may be useful in the compositions and methods of the disclosure.

By the term “detectable label” is meant, for the purposes of the specification or claims, a label molecule that is attached indirectly or directly to an antibody or antigen-binding fragment thereof according to the disclosure, wherein the label molecule facilitates the detection of the antibody in which it is incorporated. Thus, “detectable label” is used synonymously with “label molecule”.

The term “imaging agent” as used herein refers to a labeling moiety that is useful for providing an indication of the position of the label and adherents thereto, in a cell or tissue of an animal or human subject, or a cell or tissue under in vitro conditions. While agents may include those that provide detectable signals such as fluorescence, luminescence, radioactivity, or can be detected by such methods as MRI imaging, and the like, in the context of the probes and methods of use of the disclosure, the term “imaging agent” particularly refers to a label detectable by such as PET or SPECT imaging technology such as, but not limited to, ⁶⁴Cu, ⁶⁷Cu, ⁸⁹Zr, ¹²⁴I, ⁸⁶Y, ⁹⁰Y,₁₁₁In, ^{123/131}I, ¹⁷⁷Lu, ¹⁸F, ^{99m}Tc, and the like. In the most preferred embodiments of the immunoconjugate probes of the disclosure the labeling agent is 89-zirconium (⁸⁹Zr) although it is contemplated that any metal isotope (or any other PET-compatible labeling agent) may be used that provides a PET-generated image and may be attached or conjugated to the glypican-3 targeting antibody or antibody fragment.

The term “biological sample” refers to a sample obtained from an organism (e.g., a human patient) or from components (e.g., cells) of an organism. The sample may be of any biological tissue or fluid. The sample may be a “clinical sample” which is a sample derived from a patient. Such samples include, but are not limited to, sputum, blood, blood cells (e.g., white cells), amniotic fluid, plasma, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample.”

The term “tumor” as used herein refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. In particular, the probes and compositions of the disclosure are most advantageous for the detection of a cancer cells of the liver (hepatocellular carcinoma) and especially of such cells bearing epitopes of the glypican-3 membrane-bound protein.

### Abbreviations

HCC, hepatocellular carcinoma; PET, positron emission tomography; mAb, monoclonal antibody; DFO, derferoxamine; p.i., post injection.

### Description

The disclosure encompasses embodiments of probes, and methods of use thereof, for the specific detection by such technology as PET of glypican-3 (GPC3), a membrane protein that is over-expressed in over 50% of hepatocellular carcinoma (HCC) patients. In most advantageously, the probes of the disclosure comprise a glypican-3 specific antibody conjugated to a detectable label that is suitable for detection by PET. Most useful in the probes of the disclosure are anti-glypican-3-specific monoclonal antibodies or fragments thereof that retain their ability to specifically bind to an epitope of the glypican-3 protein. The detectable labels most advantageous for incorporation into the probes of the disclosure are radionuclides that are detectable by PET technology such as 89-zirconium or 64-copper. It is further contemplated that the detectable label may be attached to the glypican-3-specific moiety by a linker conjugated covalently to the immunoglobulin, the linker then receiving and retaining the label. For example, but not intended to be limiting, the linker can be a chelator having affinity for metallic entities such as 89-zirconium or 64-copper.

The affinity and specificity of an anti-GPC3 monoclonal antibody (mAb) was first determined in vitro using human recombinant GPC3 protein and human HCC cell lines. The immune-PET probe ⁸⁹Zr-DFO-GPC3 was synthesized and evaluated both in vitro and in vivo for specific cell uptake in HCC cell lines expressing varying levels of GPC3. Tumor uptake of ⁸⁹Zr-DFO-GPC3 was further evaluated in vivo using quantitative PET imaging and biodistribution analysis using nude mice bearing HCC and non-HCC xenografts in both subcutaneous and/or orthotopic tumor models. The disclosure, therefore, provides a means of specifically detecting a marker (GPC3) associated with at least 50% of hepatocellular carcinomas. More particularly, the probes of the disclosure include a detectable label that allows for the generation of a PET image that can localize cells expressing the GPC3 relative to the body of a subject animal or human. Accordingly, it becomes possible to determine the location of hepatocellular carcinoma within a subject.

An anti-GPC3 mAb has been shown to have high binding affinity to recombinant human GPC3 protein (K_{D=}0.4057±0.04562 nM), and specifically identified HCC cell lines with varying levels of GPC3 expression. In vitro, ⁸⁹Zr-DFO-GPC3 is specifically taken up, and internalized by GPC3-positive cells only. In vivo, ⁸⁹Zr-DFO-GPC3 specifically accumulates in GPC3-positive HCC xenografts but not in GPC3-negative non-HCC xenografts, with tumor-to-liver ratios of 4.01±0.17 in HepG2 cells, and only 0.29±0.08 in PC3 cells at 168 h p.i. Importantly, ⁸⁹Zr-DFO-GPC3 distinctly delineated orthotopic HCC xenografts from the surrounding normal liver, with tumor-to-liver ratios of 6.65±1.33 for HepG2, 6.15±1.75 for PLC/PRF/5, and 4.29±0.52 for Hep3B xenografts.

It has been demonstrated, therefore, that GPC3 is a viable molecular target for diagnostic imaging of HCC, and that ⁸⁹Zr-DFO-GPC3 is a clinically useful immune-PET probe for the specific and high resolution imaging of GPC3-positive HCCs. The probes and methods of the disclosure, therefore, are suitable for the early detection of HCC, allow more timely and effective clinical intervention, and thereby lead to improvements to patient survival times.
High Affinity anti-GPC3-mAb is specific for human GPC3 in vitro and in vivo: To confirm the feasibility of using GPC3 as molecular target for the diagnostic imaging of HCC based on GPC3 protein expression, the binding affinity of an anti-GPC3 mAb to recombinant human GPC3 protein was determined using an ELISA-based procedure as described by Butler et al., (1986) Mol. Immunol. 23: 971-982, and incorporated herein by reference in its entirety.

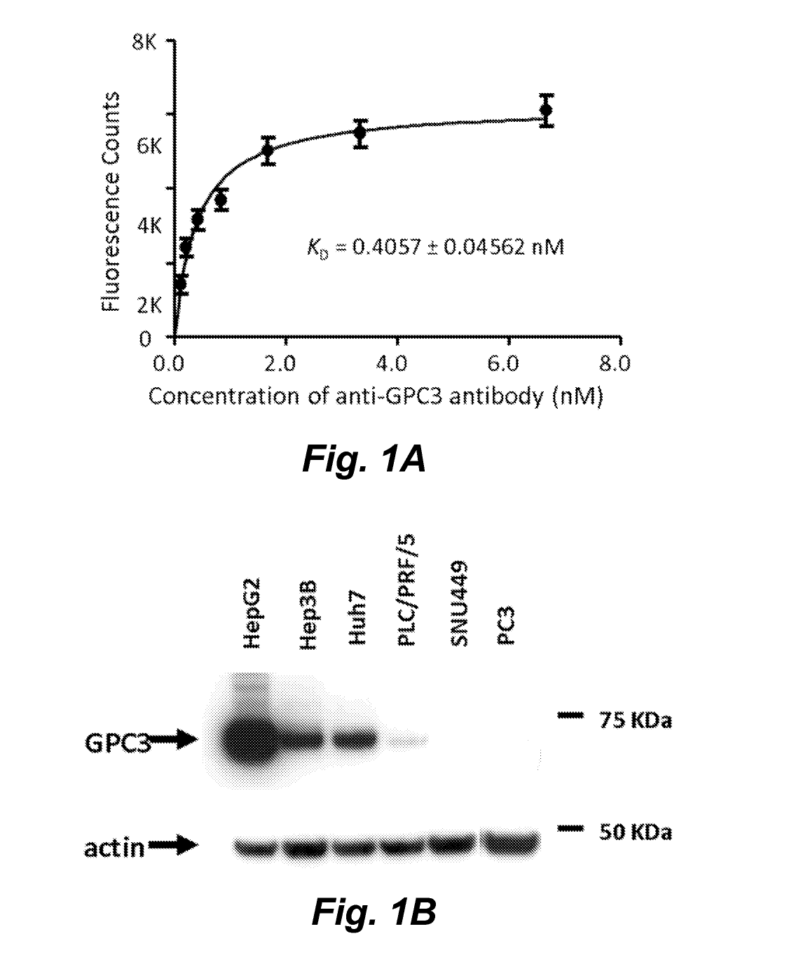

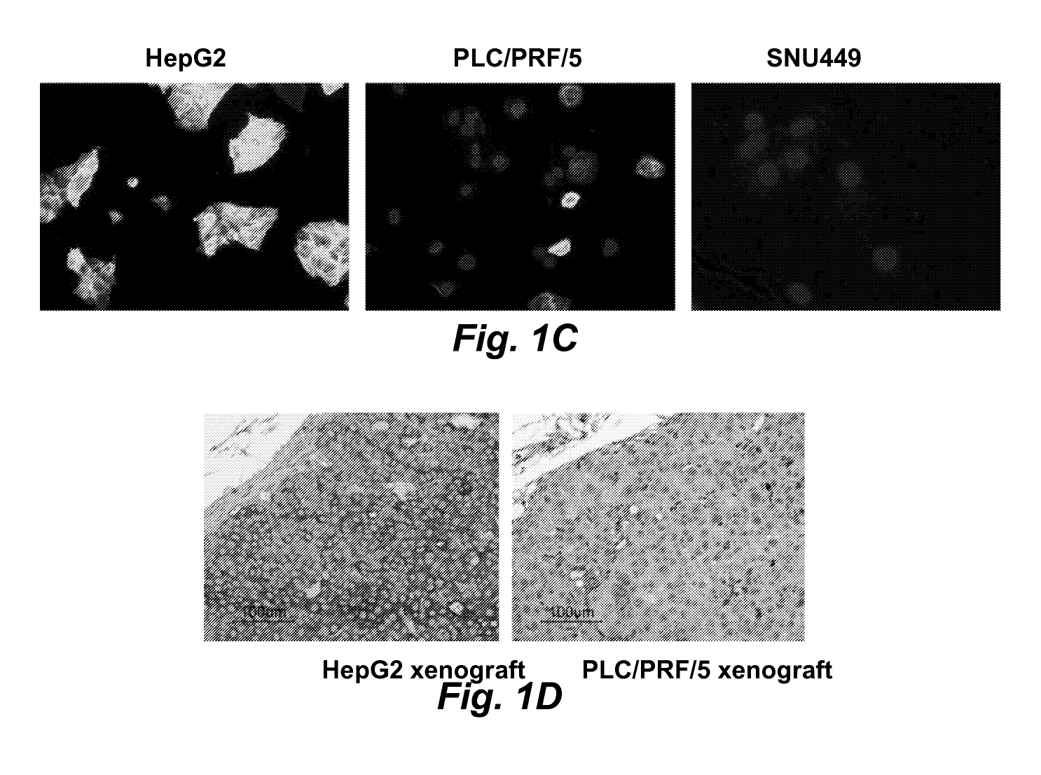

Serial dilutions of anti-GPC3 mAb caused a corresponding decrease in fluorescence signals, implying specific binding of the anti-GPC3 mAb to recombinant human GPC3 protein. The addition of the secondary fluorescence antibody alone (in the absence of GPC3 protein) did not produce a fluorescence signal above that of the background (recombinant protein only). A mean K_{D} value of 0.4057±0.04562 nM was observed, as shown in FIG. 1A, indicating high binding affinity between the recombinant human GPC3 protein and the anti-GPC3 mAb.

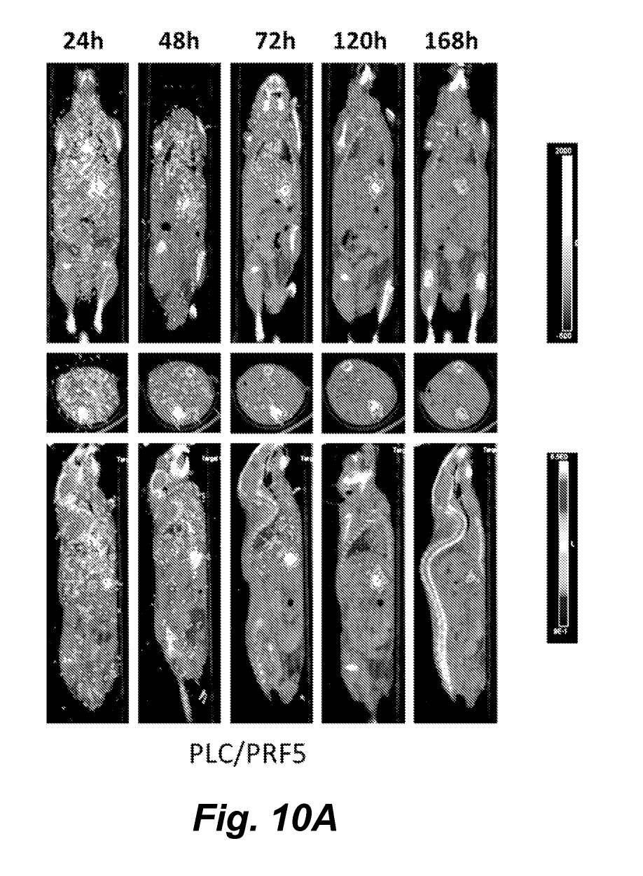

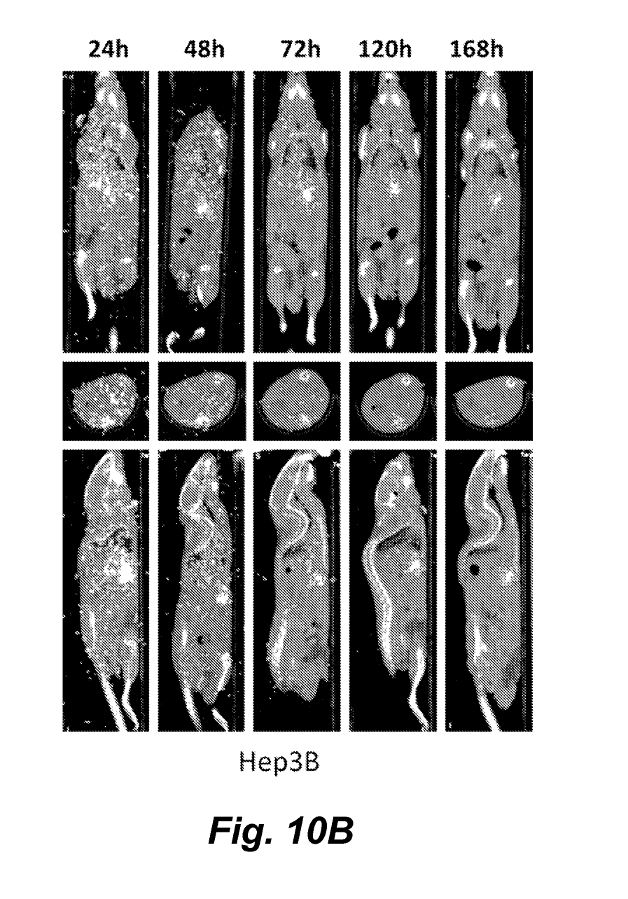

The specificity of the anti-GPC3 mAb for GPC3 protein was assessed using a panel of HCC cell lines (HepG2, Hep3B, Huh 7, PLC/PRF/5 and SNU449) and the non-HCC cell line (PC3). These cell lines showed varying levels of endogenous GPC3 protein expression, with highest levels observed in HepG2 cells, and undetectable levels in SNU449 and PC3 cells, as shown in FIG. 1 B. Immunofluorescence further confirmed the specificity of anti-GPC3 mAb, showing high fluorescence intensity in HepG2 cells, low fluorescence intensity in PLC/PRF/5 cells, and no signal in SNU449 cells, as shown in FIG. 10. Based on these results, HepG2 and PLC/PRF/5 cells were selected to represent GPC3-high and GPC3-low HCC models for further in vitro and in vivo studies. The tumorigenic PC3 cells were used as GPC3-negative, non-HCC models. Western blot and IHC detection of GPC3 protein expression in the HepG2 and PLC/PRF/5 xenografts demonstrated that the in vitro GPC3 expression patterns of the respective cell lines were maintained in vivo as shown in FIG. 1D.

Although the experiments were conducted with a single, commercially available, monoclonal antibody, it is considered within the scope of the disclosure for any anti-GPC3 antibody to be useful in the probe compositions of the disclosure. It is further contemplated that it would be advantageous to prepare from any such monoclonal antibody a fragment thereof that has retained the GPC3-specific binding ability of the original monoclonal antibody immunoglobulin. It is further contemplated that the probe compositions of the present disclosure, particularly if combined with a suitable pharmaceutically acceptable carrier, may be a mixture of anti-GPC3 antibodies, or fragments thereof, wherein each antibody may have specific affinity for a particular epitope of the GPC3.
Specific Uptake of ⁸⁹Zr-DFO-GPC3 into human HCC cells expressing GPC3: The ⁸⁹Zr-DFO-GPC3 PET probe was synthesized and its cellular uptake and internalization into a panel of human HCC cell lines and non-HCC cell lines with varying levels of endogenous GPC3 expression was assessed. It is, however, contemplated to be within the scope of the disclosure for linkers other than DFO to be used in the generation of the probes herein disclosed, including linkers that have metal chelating properties.

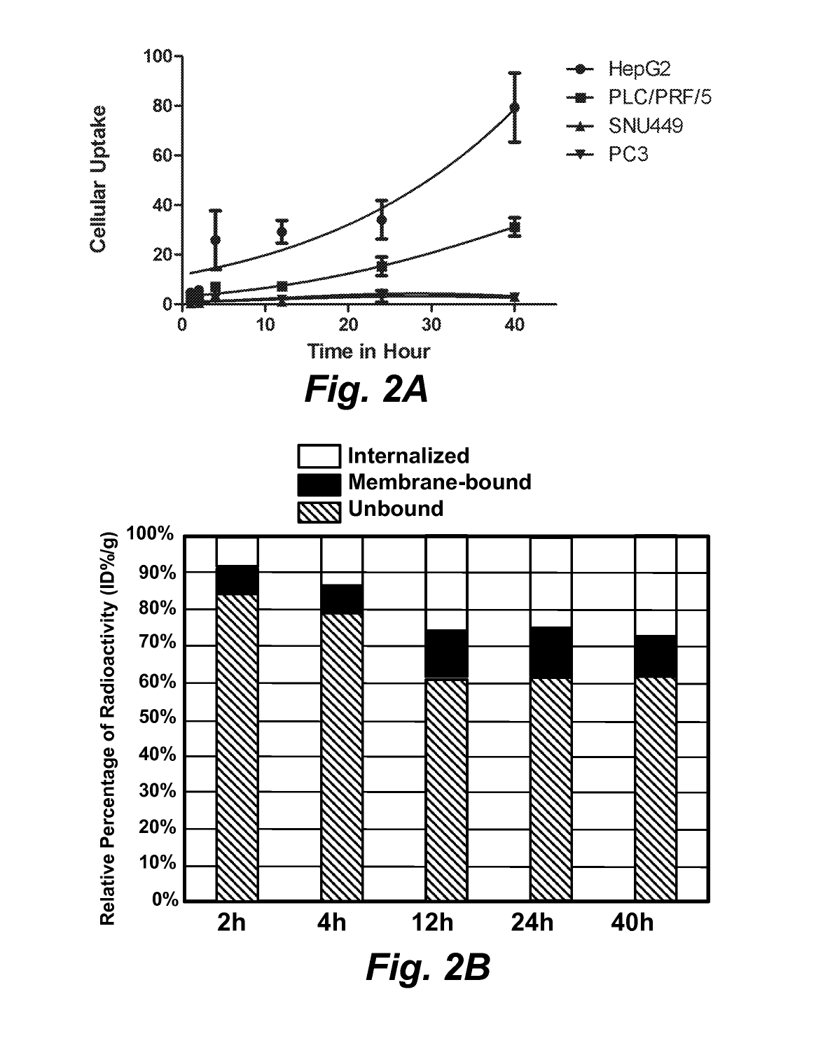

It was found that the overall uptake of ⁸⁹Zr-DFO-GPC3 into these cell lines corresponded with the level of GPC3 expression in these cells, i.e., the highest cellular uptake was observed in HepG2 cells with the highest level of GPC3 expression, with uptake being significantly higher than in all other cell lines at every time point, as shown in FIG. 2A. Moderate cellular uptake was observed in PLC/PRF/5 cells with low GPC3 expression, whereas negligible uptake was observed in the GPC3-negative SNU449, A375M, and PC3 cells. At 40 h, cellular uptake in HepG2 was 79.30±13.94 (% ID/g (n=4), compared to 31.21±3.65 (% ID/g in PLC/PRF/5 (n=4), 3.13±0.36% ID/g in SNU449 (n=4), and 2.64±0.60 (% ID/g in PC3 cells. These data demonstrate that ⁸⁹Zr-DFO-GPC3 can be taken up specifically into GPC3-expressing cells.

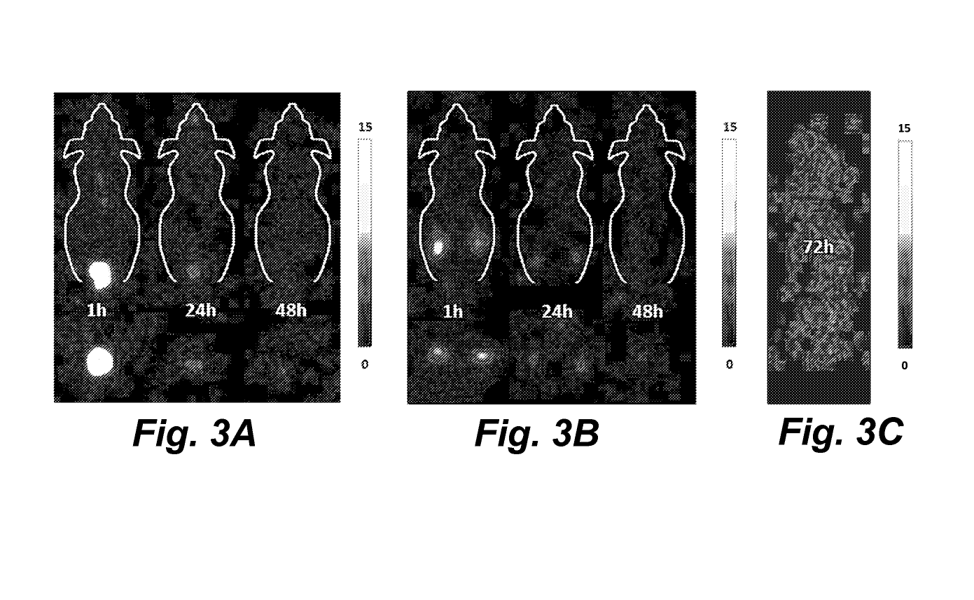

Internalization of ⁸⁹Zr-DFO-GPC3 was also observed in GPC3-positive HepG2 cells, as shown in FIG. 2B. About 8.64±0.58% of the added radioactivity was detected in the internalized fraction 2 h post-incubation, which slowly increased to 27.42±1.38% at 40 h post-incubation. The percentage of the internalized fraction was higher than that of the membrane-bound fraction at every time point, with significance (p<0.05) observed from 12 h onwards. Furthermore, the ratio of internalized vs. total bound (internalized and membrane) percentages did not change significantly after 12 h.
Distribution of free ⁸⁹Zr ions in mice: To eliminate the possibility that free ⁸⁹Zr might accumulate in the liver and lead to high liver signals, free ⁸⁹Zr ions were injected into a normal mouse for PET imaging that was performed starting at 1 h post-injection (p.i). The greatest signal was observed in the bladder and kidneys of the mice at 1 h p.i, as shown in FIGS. 3A and 3B. Within 24 h the majority of free ⁸⁹Zr ions was cleared out through the urinary tract, leaving only trace levels of radioactivity in the bladder and kidneys. At 48 h, there was minimal radioactivity detected in these tissues. Minimal accumulation of radioactivity in the bone only was observed at 72 h p.i, as shown in FIG. 3C, consistent with what was known of ⁸⁹Zr ions (Zhang et al., (2011) Curr. Radiopharm. 4: 131-139. Liver accumulation of ⁸⁹Zr was not detected at any time point.
⁸⁹Zr-DFO-GPC3 specifically identifies subcutaneous HCC xenografts expressing GPC3: ⁸⁹Zr-DFO-GPC3 radiotracer was used for PET imaging of subcutaneous xenografts generated using HepG2 (GPC3-high expression, HCC cells), PLC/PRF/5 (GPC3-low expression, HCC cells) and PC3 cells (GPC3-negative, non-HCC cells) (the GPC3-negative HCC cell line SNU449 was found to be non-tumorigenic in mice).

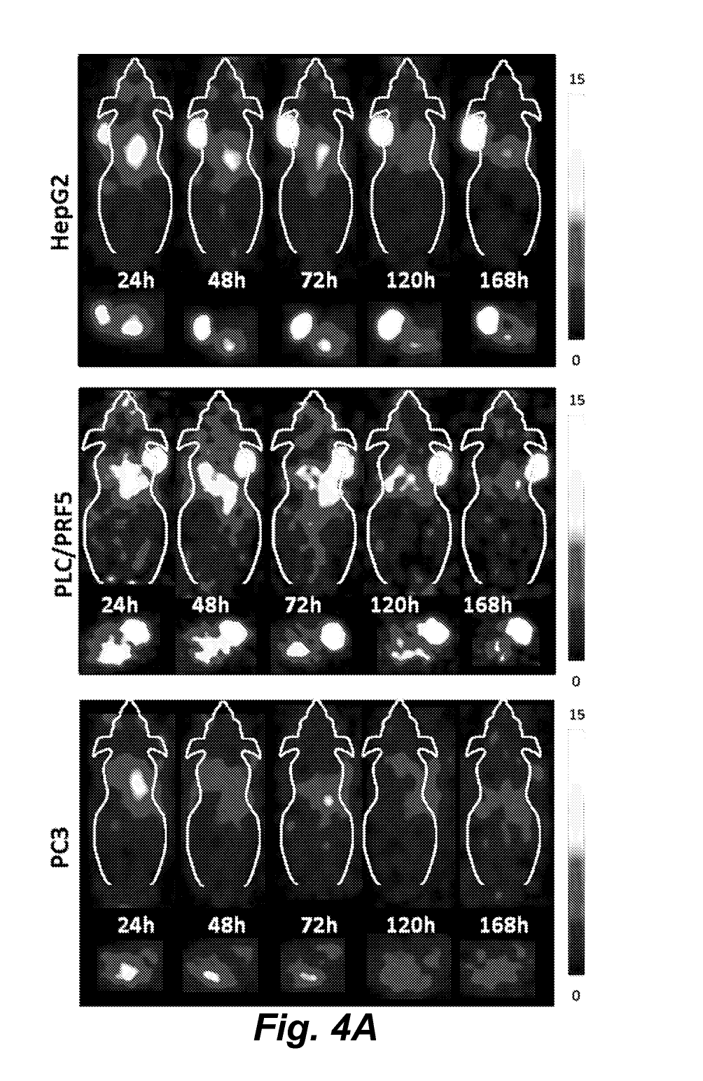

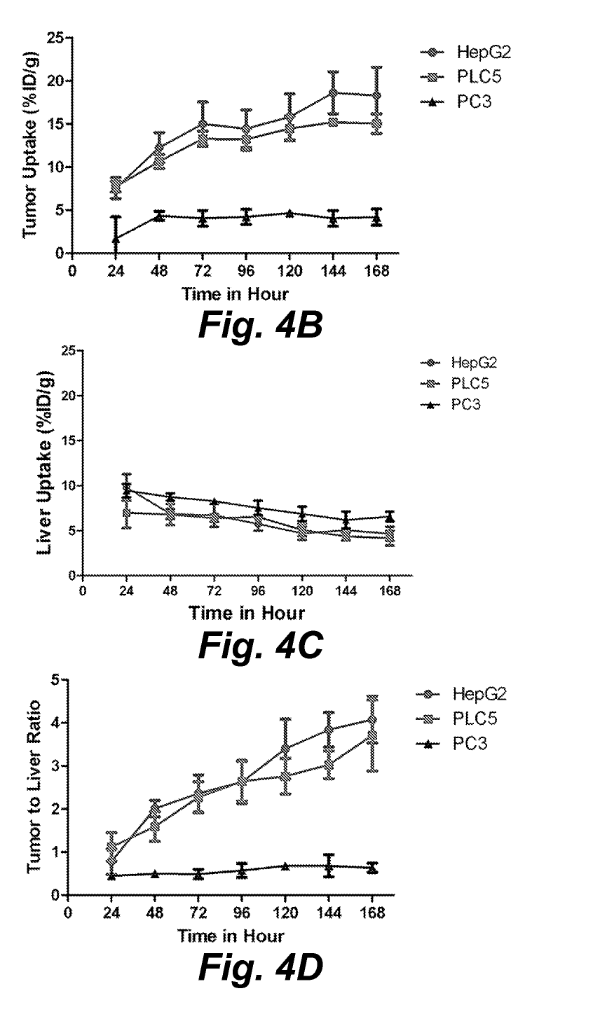

The specificity of the radiotracer for GPC3-expressing xenografts was demonstrated from the decay-corrected coronal and transaxial small-animal PET images in the tumor-bearing mice after injection of ⁸⁹Zr-DFO-GPC3, as shown in FIG. 4A. ⁸⁹Zr-DFO-GPC3 clearly delineated GPC3-expressing HCC xenografts regardless of their endogenous level of GPC3. Both HepG2 xenografts (top panel) and PLC/PRF/5 xenografts (middle panel) showed increasing tumor uptake over time. Non-specific liver signals in both the HepG2 and PLC/PRF/5 xenograft-bearing mice, however, were observed to be highest at 24 h p.i and decreased over time.

In mice bearing PC3 xenografts, minimal tumor signal was observed, again indicating specificity of the radiotracer for GPC3-expressing xenografts only. Liver uptake in mice bearing PC3 xenografts showed a similar pattern as mice bearing HepG2 xenografts, with the highest signal at 24 h p.i, and which decreased over time.

Quantification analysis revealed significantly higher tumor uptake in HepG2 and PLC/PRF/5 xenografts compared to PC3 xenografts (p<0.05), starting at 48 h p.i, as shown in FIG. 4B. For example, the tumor uptake of HepG2 and PLC/PRF/5 xenografts at 48 h p.i was 12.27±1.73% ID/g and 10.66±0.81% ID/g respectively, compared with 4.34±0.53% ID/g for PC3 xenografts. Tumor uptakes in HepG2 and PLC/PRF/5 xenografts increased over time, to the highest levels of 18.31±3.28% ID/g and 15.05±1.14% ID/g at 168 h p.i, respectively. Tumor uptake in PLC/PRF/5 xenografts also increased over time, to the highest level of at 168 h p.i. Tumor signals in PC3 xenografts did not increase over time (FIG. 4B). The liver uptakes in all three xenograft models were similar at all time points, and all decreased over time, as shown in FIG. 4C. The tumor-to-liver ratios in HepG2 and PLC/PRF/5 tumor-bearing mice increased steadily over time, from 2.01±0.19 at 48 h p.i to 4.08±0.54 at 168 h p.i for HepG2, and from 1.59±0.34 at 48 h p.i to 3.71±0.83 at 168 h p.i for PLC/PRF/5. In contrast, the tumor-to-liver ratios for PC3 xenografts remained at about 1.0, indicating similar uptake into the GPC3-negative xenograft and the liver, as shown in FIG. 4D.

These results are supported by in vivo biodistribution analysis as shown in Table 1, which showed significantly higher uptake in HepG2 xenografts (13.14±1.68% ID/g, n=4) and PLC/PRF/5 xenografts (12.18±0.90% ID/g, n=4), compared with the PC3 (2.58±0.27% ID/g) xenografts at 168 h p.i (p<0.005).

TABLE 1
Biodistribution of ⁸⁹Zr-DFO-GPC3 Tracers in Subcutaneous Models

| ⁸⁹Zr-DFO-GPC3 | HepG2 (48 h) | HepG2 | PLC/PRF/5 | PC3 |
|---|---|---|---|---|
| Tissues (% ID/g) | | | | |
| Tumor | 15.12 ± 0.69* | 13.14 ± 1.68* | 12.18 ± 0.90* | 2.58 ± 0.27 |
| Blood | 10.50 ± 1.64 | 2.50 ± 0.54 | 2.71 ± 0.57 | 4.82 ± 0.07 |
| Heart | 3.27 ± 0.63 | 0.83 ± 0.18 | 0.83 ± 0.12 | 1.46 ± 0.14 |
| Lungs | 4.59 ± 0.86 | 1.80 ± 0.21 | 1.60 ± 0.38 | 2.86 ± 0.14 |
| Liver | 6.32 ± 1.95 | 3.20 ± 0.34 | 3.37 ± 0.07 | 2.34 ± 0.08 |
| Spleen | 3.94 ± 0.37 | 2.29 ± 0.35 | 2.46 ± 0.65 | 3.52 ± 0.33 |
| Pancreas | 1.29 ± 0.23 | 0.37 ± 0.10 | 0.35 ± 0.09 | 0.72 ± 0.04 |
| Stomach | 1.34 ± 0.20 | 0.40 ± 0.05 | 0.37 ± 0.04 | 0.70 ± 0.01 |
| Brain | 0.37 ± 0.05 | 0.09 ± 0.01 | 0.13 ± 0.02 | 0.20 ± 0.01 |
| Intestine | 1.51 ± 0.35 | 0.42 ± 0.04 | 0.47 ± 0.24 | 0.58 ± 0.15 |
| Kidneys | 3.19 ± 0.67 | 1.18 ± 0.10 | 1.21 ± 0.32 | 1.76 ± 0.11 |
| Skin | 3.40 ± 0.68 | 1.15 ± 0.23 | 0.87 ± 0.75 | 2.22 ± 0.24 |
| Muscle | 0.82 ± 0.11 | 0.26 ± 0.02 | 0.23 ± 0.02 | 0.38 ± 0.04 |
| Bone | 2.87 ± 0.27 | 2.09 ± 0.32 | 1.94 ± 0.76 | 2.12 ± 0.66 |
| Uptake Ratio of | | | | |
| Tumor/Normal Tissue | | | | |
| Tumor/Liver | 2.39 ± 0.19* | 4.10 ± 0.17* | 3.61 ± 0.14* | 1.10 ± 0.27 |
| Tumor/Muscle | 18.51 ± 1.52* | 49.10 ± 1.97* | 52.14 ± 1.79* | 6.92 ± 1.34 |

*p < 0.05 (n = 4), significant difference, compared with PC3 tumors

Data are given as mean±SD of percentage administered activity (injected dose) per gram of tissue (% ID/g).

The tumor-to-liver ratios at 168 h p.i reached 4.10±0.17 in HepG2 xenografts, and 3.61±0.14 in PLC/PRF/5 xenografts, both of which were significantly higher than that in PC3 xenografts (2.58±0.08) (p<0.005). Biodistribution analysis at an earlier time point (48 h p.i) showed that a good tumor-to-liver ratio was achieved in HepG2 xenografts (2.39±0.19% ID/g). These results indicate that ⁸⁹Zr-DFO-GPC3 can specifically identify GPC3-expressing tumors.
⁸⁹Zr-DFO-GPC3 specifically identifies orthotopic HCC xenografts expressing GPC3: To provide a clinically relevant model to assess the ability of ⁸⁹Zr-DFO-GPC3 to delineate liver tumors from the surrounding non-tumor liver, orthotopic HCC xenografts were generated from HCC cell lines that expressed varying levels of endogenous GPC3 (HepG2, Hep3B, and PLC/PRF/5, n=4 for each group).

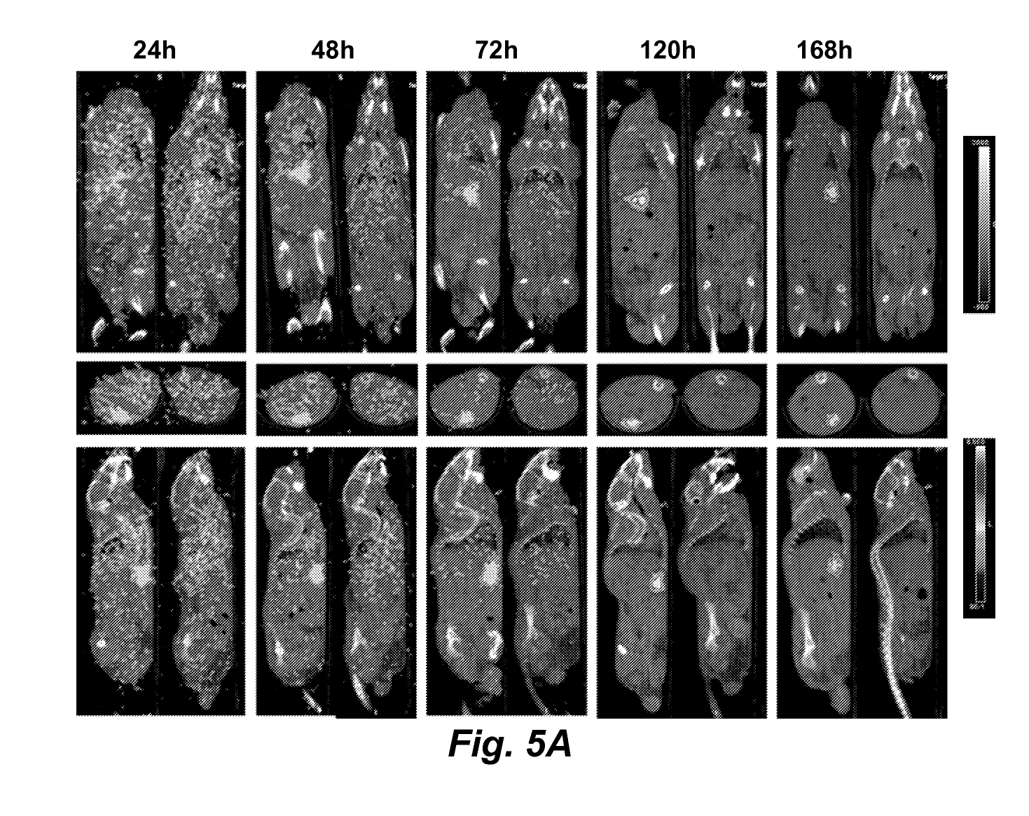

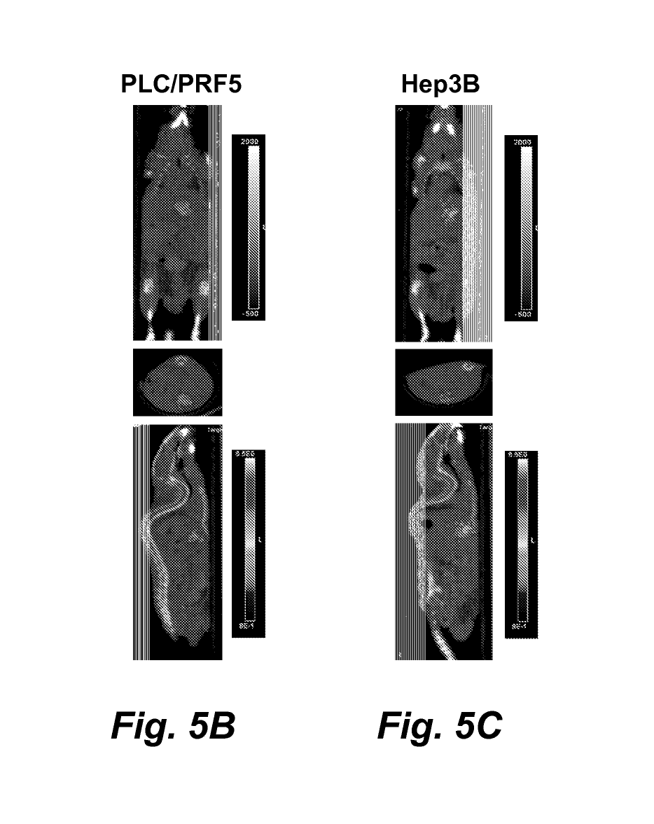

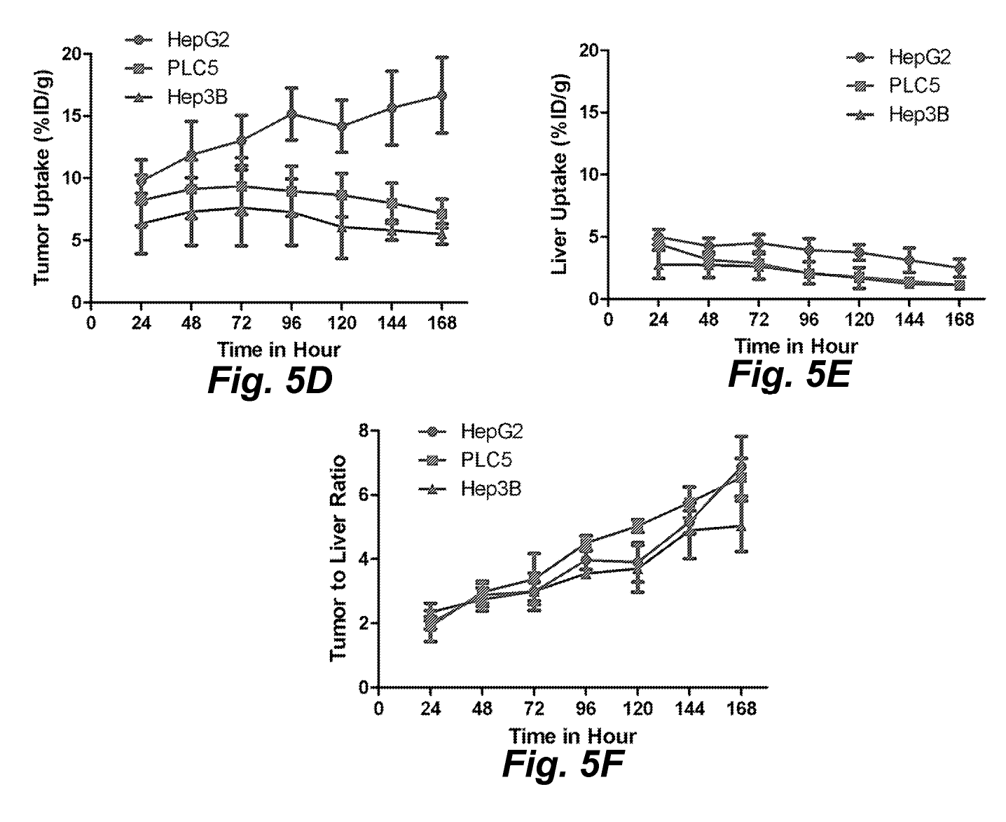

As controls, normal mice (n=4) were injected with the same amount of ⁸⁹Zr-DFO-GPC3, and imaged at the same time points. Decay-corrected coronal, transaxial, and sagittal views of PET/CT images in HepG2 tumor-bearing mice and normal mice are shown in FIG. 5A. Similar to the observations with the subcutaneous xenografts, the radiotracer was observed to accumulate over time in the HepG2 tumors, whereas the liver signals decreased over time, allowing distinct delineation of the tumor from the liver at the final scan time of 168 h p.i. The liver within the tumor-bearing mice showed negligible radiotracer uptake, consistent with the normal mouse control. Similar trends were observed in animals bearing orthotopic Hep3B, as shown in FIG. 5B and PLC/PRF/5 (FIG. 5C) xenografts. PET/CT quantification analysis revealed increasing radiotracer uptake in HepG2 xenografts, which reached 16.67±3.04% ID/g at 168 h p.i. Tumor uptake in Hep3B and PLC5/PRF/5 xenografts was lower than in HepG2 xenografts, and slightly decreased over time (7.27±0.83% ID/g for Hep3B, and 8.63±1.15% ID/g for PLC/PRF/5 at 168 h p.i). While not being bound to any one theory, this result may be in part due to the lower levels of GPC3 expression in these tumors and to radioactivity decay over time (see FIG. 5D).

Liver uptakes in the mice of all three xenograft models were similarly low and decreased over time (FIG. 5E). All three orthotopic HCC xenograft models show high tumor-to-liver ratios (6.88±0.95 for HepG2, 6.55±0.59 for PLC/PRF/5 and 5.03±0.79 for Hep3B) (FIG. 5F). In vivo biodistribution data consistently showed high tumor-to-liver ratios in all three orthotopic HCC xenografts, with 6.65±2.33 for HepG2, 6.15±1.75 for PLC/PRF/5, and 4.29±0.52 for Hep3B, as shown in Table 2.

TABLE 2
Bio Distribution of ⁸⁹Zr-DFO-GPC3 Tracers in Orthotopic Models

| ⁸⁹Zr-DFO-GPC3 | HepG2 | PLC/PRF/5 | Hep3B | Normal |
|---|---|---|---|---|
| Tissues (% ID/g) | | | | |
| Tumor | 14.93 ± 3.50 | 7.71 ± 1.64 | 6.54 ± 1.00 | N/A |
| Blood | 1.13 ± 0.95 | 2.12 ± 0.19 | 2.09 ± 0.59 | 1.96 ± 0.62 |
| Heart | 0.35 ± 0.19 | 0.67 ± 0.10 | 0.58 ± 0.19 | 0.60 ± 0.18 |
| Lungs | 0.62 ± 0.29 | 1.16 ± 0.07 | 0.98 ± 0.21 | 1.06 ± 0.29 |
| Liver | 2.50 ± 1.03 | 1.27 ± 0.12 | 1.31 ± 0.31 | 0.92 ± 0.14 |
| Spleen | 1.13 ± 0.12 | 1.94 ± 0.18 | 1.01 ± 0.56 | 1.07 ± 0.36 |
| Pancreas | 0.23 ± 0.14 | 0.36 ± 0.02 | 0.33 ± 0.09 | 0.28 ± 0.08 |
| Stomach | 0.28 ± 0.12 | 0.36 ± 0.03 | 0.31 ± 0.13 | 0.23 ± 0.08 |
| Brain | 0.05 ± 0.04 | 0.09 ± 0.02 | 0.07 ± 0.01 | 0.08 ± 0.01 |
| Intestine | 0.19 ± 0.10 | 0.48 ± 0.07 | 0.33 ± 0.11 | 0.32 ± 0.14 |
| Kidneys | 0.56 ± 0.14 | 1.00 ± 0.13 | 0.79 ± 0.17 | 0.73 ± 0.16 |
| Skin | 0.78 ± 0.25 | 0.89 ± 0.04 | 1.08 ± 0.10 | 0.87 ± 0.35 |
| Muscle | 0.27 ± 0.05 | 0.20 ± 0.01 | 0.21 ± 0.06 | 0.21 ± 0.05 |
| Bone | 1.55 ± 0.71 | 1.41 ± 0.03 | 1.13 ± 0.23 | 1.07 ± 0.31 |
| Uptake Ratio of | | | | |
| Tumor/Normal Tissue | | | | |
| Tumor/Liver | 6.65 ± 2.33 | 6.15 ± 1.75 | 4.29 ± 0.52 | |
| Tumor/Muscle | 55.30 ± 2.96 | 38.34 ± 2.62 | 25.67 ± 3.28 | |

Data are given as mean±SD of percentage administered activity (injected dose) per gram of tissue (% ID/g).

The accumulation of data, therefore, indicates that the ⁸⁹Zr-DFO-GPC3 radiotracer can clearly differentiate tumor lesions from their surrounding non-tumor liver, and suggest potential clinical usefulness of this probe.

The present disclosure provides for the synthesis of ⁸⁹Zr-labeled monoclonal antibody against human GPC3, a membrane protein over-expressed in a large percentage of HCC patients, and demonstrated its ability to specifically identify GPC3-expressing HCC cells in vitro and in vivo. It also distinctly delineated GPC3-expressing HCC orthotopic xenografts from surrounding non-tumor liver, suggesting the potential for clinical translation of this probe.

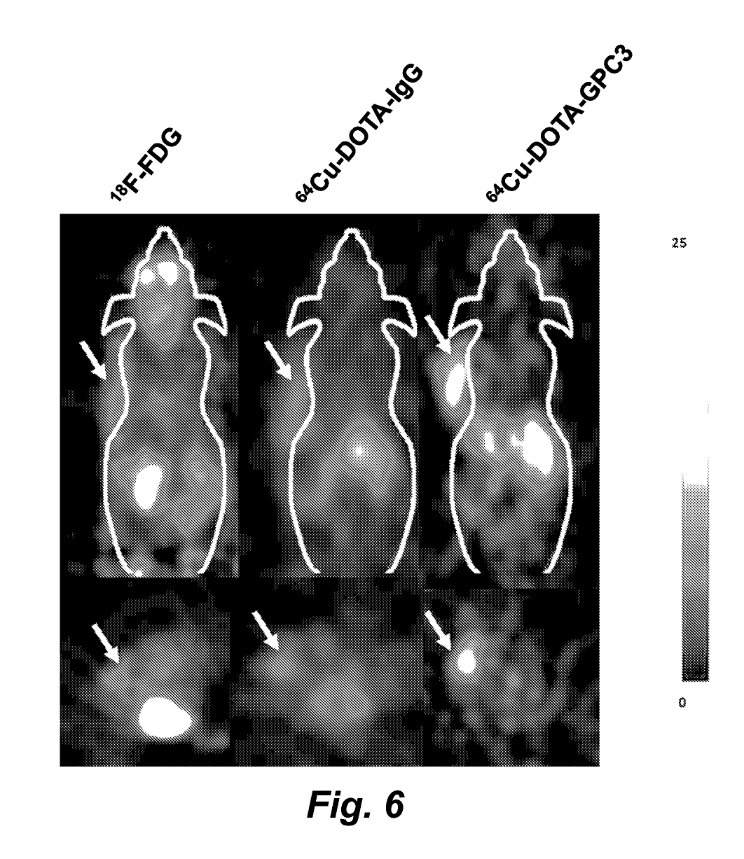

An earlier ⁶⁴Cu-DOTA-GPC3 probe was superior to the more commonly used PET tracer ¹⁸F-FDG in detecting GPC3-positive HCC xenografts, as shown in FIG. 6. FDG-PET showed very weak signals in HepG2 xenografts, which were only slightly higher than that from the non-specific control ⁶⁴Cu-DOTA-anti-IgG. FDG-PET is commonly used for the diagnosis, staging, and monitoring treatment of various cancers, since ¹⁸F-FDG is taken up by cells, phosphorylated by hexokinase, and retained by tissues with high metabolic activity, such as most types of malignant tumors (Fowler & Ido (2002) Semin. Nucl. Med. 32: 6-12).

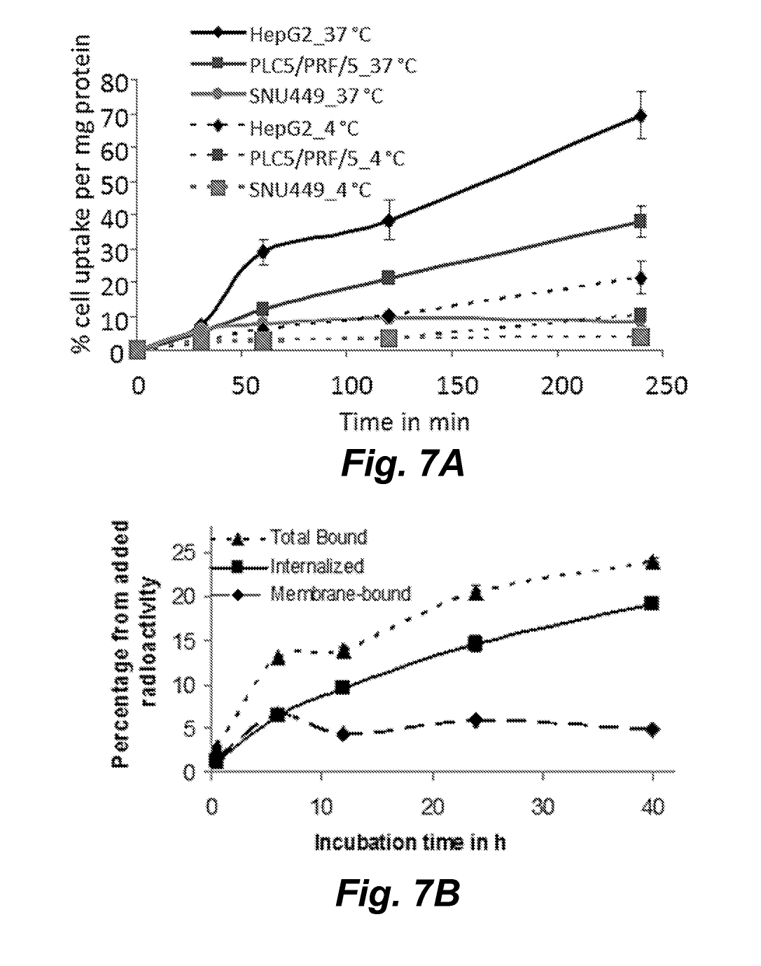

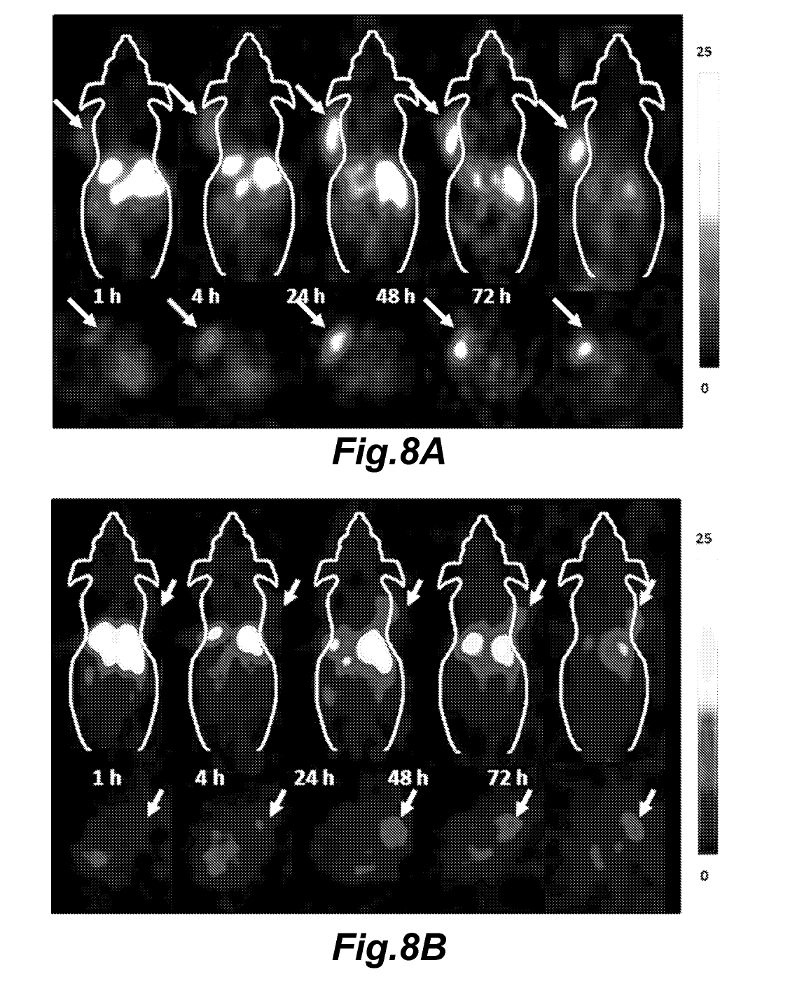

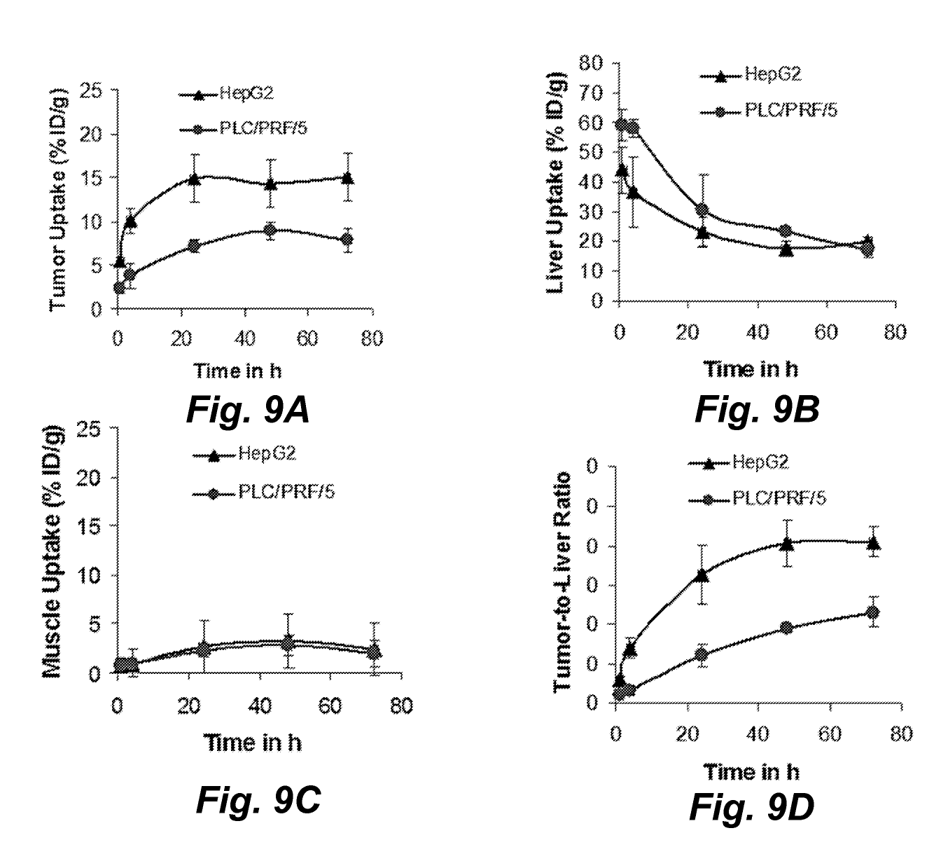

In the detection of HCC, ¹⁸F-FDG-PET remains challenging (and misses 30-50% of HCCs) because of the inherent background from metabolic activities in the region of interest. It has also been shown that ⁶⁴Cu-DOTA-GPC3 can be specifically taken up by, and internalized within, HCC cells expressing GPC3, as shown in FIGS. 7A and 7B. While ⁶⁴Cu-DOTA-GPC3 is a more specific probe than ¹⁸F-FDG, its clinical use is limited by the low tumor-to-liver ratios (0.46±0.32 in HepG2 tumors at 48 h p. i.) resulting from high liver uptake and the short half-life of ⁶⁴Cu, as shown in FIGS. 8A and 8B and in Table 3.

TABLE 3
Bio Distribution of ⁶⁴Cu-DOTA Tracers in Subcutaneous Models

| Tissue | HepG2 tumors | | PLC/PRF/5 tumors |
|---|---|---|---|
| Tissues (% ID/g) | ⁶⁴Cu-DOTA-GPC3 | ⁶⁴Cu-DOTA-IgG | ⁶⁴Cu-DOTA-GPC3 |
| Tumor | 17.05 ± 1.31*\hat{ } | 5.10 ± 1.27 | 11.63 ± 1.19\hat{ } |
| Blood | 3.59 ± 0.74 | 4.18 ± 0.45 | 3.94 ± 0.21 |
| Heart | 6.38 ± 1.81 | 3.08 ± 0.47 | 5.82 ± 0.38 |
| Liver | 18.75 ± 0.53\hat{ } | 6.67 ± 0.83 | 23.34 ± 3.45\hat{ } |
| Lungs | 6.91 ± 0.10 | 3.43 ± 0.30 | 6.83 ± 1.27 |
| Muscles | 0.69 ± 0.07 | 0.54 ± 0.15 | 0.78 ± 0.20 |
| Spleen | 4.05 ± 0.73 | 2.28 ± 0.20 | 6.13 ± 2.47 |
| Brain | 0.74 ± 0.05 | 0.61 ± 0.29 | 0.81 ± 0.06 |
| Skin | 2.13 ± 0.71 | 2.04 ± 0.11 | 3.04 ± 0.28 |
| Stomach | 5.56 ± 1.08 | 1.15 ± 0.15 | 4.49 ± 2.09 |
| Pancreas | 2.29 ± 0.47 | 1.21 ± 0.27 | 3.67 ± 1.91 |
| Bone | 1.43 ± 0.55 | 1.31 ± 0.89 | 1.44 ± 0.60 |
| Kidneys | 8.04 ± 1.54 | 4.73 ± 0.74 | 7.67 ± 0.21 |
| Intestine | 1.93 ± 0.38 | 1.92 ± 1.28 | 2.51 ± 0.49 |
| Tail | 2.21 ± 1.35 | 1.05 ± 0.08 | 2.20 ± 0.13 |
| Uptake Ratio of | | | |
| Tumor/Normal Tissue | | | |
| Tumor/Blood | 4.92 ± 1.21*\hat{ } | 1.21 ± 0.25 | 2.96 ± 0.41\hat{ } |
| Tumor/Muscle | 24.95 ± 4.05*\hat{ } | 9.91 ± 3.51 | 15.30 ± 2.96 |
| Tumor/Liver | 0.46 ± 0.32 | 0.28 ± 0.20 | 0.51 ± 0.11 |

*p < 0.05 (n = 4), significant difference, compared with ⁶⁴Cu-DOTA-GPC3 in PLC/PRF/5 tumors.
\hat{ }p < 0.05 (n = 4), significant difference, compared with ⁶⁴Cu-DOTA-IgG in HepG2 tumors.

Data are given as mean±SD of percentage administered activity (injected dose) per gram of tissue (% ID/g).

Although mAbs labeled with PET- and SPECT-radioisotopes have been used clinically in the diagnosis and treatment of various solid tumors (Borjesson et al., (2009) J. Nucl. Med. 50: 1828-1836; Elsasser-Beile et al., (2009) J. Nucl. Med. 50: 606-611), the data with the ⁶⁴Cu-DOTA-GPC3 probe highlights the well-recognized drawback of high liver background when using mAbs in diagnostic imaging, which is a clinical challenge when it comes to liver tumor imaging. Accordingly, a longer half-life radioisotope, zirconium-89 (Zr⁸⁹), was selected to circumvent this problem and achieve clinically favorable tumor-to-liver ratios and had the following advantage(s): (i) the long decay half-life (3.3 day; 78.4 h) of ⁸⁹Zr matches the pharmacokinetics of intact mAb molecules; (ii) the long-lasting radioactivity allows imaging at late time points (up to seven days p.i) for obtaining maximum information; and (iii) the ability to residualize and therefore be retained within the target cell after internalization and intracellular degradation of the tracer results in enhanced uptake in the tumor when an internalized antibody is used.

The ⁸⁹Zr-DFO-GPC3 PET probe according to the disclosure demonstrated, therefore, that this probe is advantageous compared to the ⁶⁴Cu-DOTA-GPC3 probe, particularly in achieving high tumor-to-liver ratios due to enhanced tumor accumulation and reduced non-specific liver accumulation. In subcutaneous xenograft models, ⁸⁹Zr-DFO-GPC3 specifically detected GPC3-expressing HCC xenografts only, with minimal accumulation in non-GPC3 expressing, non-HCC cell lines. ⁸⁹Zr-DFO-GPC3 also distinctly delineated orthotopic HCC xenografts from the surrounding non-tumor liver when imaged seven days p.i, providing high resolution imaging of the tumor lesions. Additionally, ⁸⁹Zr-DFO-GPC3 was able to detect all three HCC xenograft models (with varying levels of GPC3 expression), implying specificity for GPC3-expressing HCCs and highlighting its clinical value in the diagnosis of all GPC3-expressing HCC lesions, regardless of GPC3 expression level.

Accordingly, it has been shown that GPC3 is suitable for molecular targeting for the diagnostic imaging of HCC, and that the ⁸⁹Zr-DFO-GPC3 probe is a clinically useful immune-PET probe for the specific and high resolution imaging of GPC3-expression HCCs. The successful imaging of HCC lesions based on GPC3 expression is advantageous for early detection of HCC, and can also allow more accurate prognostication of HCC patients, since GPC3-positive HCC patients have been reported to have significantly lower 5-year survival rate than GPC3-negative HCC patients (Wang et al., (2008) Arch. Pathol. Lab. Med. 132: 1723-1728). This offers the possibility of better patient stratification based on GPC3 expression levels, leading to improved clinical management and eventually improved patient survival rate.

One aspect of the disclosure, therefore, encompasses embodiments of an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT.

In embodiments of this aspect of the disclosure, the detectable label can be a radionuclide selected from the group consisting of: ⁶⁴Cu, ⁶⁷Cu, ₈₉Zr, ¹²⁴I, ⁸⁶Y, ⁹⁰Y, ¹¹¹In, ^{123/131}I, ¹⁷⁷Lu, ¹⁸F, and ^{99m}Tc.

In embodiments of this aspect of the disclosure, the detectable label can be detectable by positron emission tomography (PET) and is zirconium⁸⁹ (⁸⁹Zr) or copper⁶⁴ (⁶⁴Cu).

In embodiments of this aspect of the disclosure, the detectable label can be attached to the anti-GPC3-specific antibody (mAb), or a target-specific fragment thereof, by a linker.

In some embodiments of this aspect of the disclosure, the linker can be DFO.

Another aspect of the disclosure encompasses embodiments of a pharmaceutically acceptable composition comprising: an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT, and further comprising a pharmaceutically acceptable carrier.

Yet another aspect of the disclosure encompasses embodiments of a method of obtaining an image of a hepatocellular carcinoma in a subject animal or human, the method comprising the steps of: (a) delivering to a subject animal or human a pharmaceutically acceptable composition comprising an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT; (b) subjecting the subject animal or human to positron emission tomography; (c) identifying a detectable signal from the probe in the subject animal or human; and (d) generating an image of the detectable signal, thereby obtaining an image of a hepatocellular carcinoma in a subject animal or human.

In embodiments of this aspect of the disclosure, the detectable label can be zirconium⁸⁹ (⁸⁹Zr) or copper⁶⁴ (⁶⁴Cu).

In embodiments of this aspect of the disclosure, the detectable PET label can be attached to the anti-GPC3-specific antibody (mAb) or the target-specific fragment thereof by a linker. In some embodiments of this aspect of the disclosure, the linker can be DFO.

Still another aspect of the disclosure encompasses embodiments of a method of detecting a cell having glypican-3 (GPC3), or population of said cells, in a biological sample, the method comprising the steps of: (a) obtaining a biological sample from an animal or human subject; (b) contacting the biological sample with an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT; and (c) subjecting the biological sample to positron emission tomography, whereupon a detectable signal from the probe indicates the presence of a cell having glypican-3 (GPC3), or population of said cells, in the biological sample.

In embodiments of this aspect of the disclosure, the detectable label can be zirconium⁸⁹ (⁸⁹Zr).

In embodiments of this aspect of the disclosure, the detectable PET label can be attached to the anti-GPC3-specific antibody (mAb) or the target-specific fragment thereof by a linker. In some embodiments of this aspect of the disclosure, the linker can be DFO.

Still another aspect of the disclosure encompasses embodiments of a method of determining if a subject animal or human has a hepatocellular carcinoma expressing glypican-3 (GPC3), the method comprising the steps of: (a) obtaining a biological sample from an animal or human subject; (b) contacting the biological sample with an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT; (c) subjecting the biological sample to positron emission tomography; and (d) identifying a detectable signal from the probe, wherein the detection of the probe indicates the presence of a cell having glypican-3 (GPC3), or population of said cells, in the biological sample, thereby indicating the presence of a hepatocellular carcinoma in the subject animal or human.

In embodiments of this aspect of the disclosure, the detectable PET label can be zirconium⁸⁹ (⁸⁹Zr).

In embodiments of this aspect of the disclosure, the detectable PET label can be attached to the anti-GPC3-specific antibody (mAb) or the target-specific fragment thereof by a linker. In some embodiments of this aspect of the disclosure, the linker can be DFO.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt% to about 5 wt%, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include±1%, ±2%, ±3%, ±4%, ±5%, ±6%, ±7%, ±8%, ±9%, or ±10%, or more of the numerical value(s) being modified.

### EXAMPLES

### Example 1

Cell Culture: The human HCC cell lines HepG2, Hep3B, and PLC/PRF/5 and the non-HCC cell line PC3 were cultured in Dulbecco's Modified Eagle's Medium (DMEM), supplemented with 10% fetal bovine serum (FBS), and 1% penicillin-streptomycin (Invitrogen Life Technologies, Carlsbad, Calif.). The HCC cell line SNU449 was cultured in RPMI-1640 medium with 10% FBS and 1% penicillin-streptomycin. All cell lines were maintained in a humidified atmosphere of 5% CO₂ at 37° C.

### Example 2

Affinity Assay: The binding affinity of anti-GPC3 mAb (Clone 1 G12, BioMosaics Inc., Burlington, Vt.) to recombinant human GPC3 protein was determined using an ELISA-based affinity binding assay as described in Butler et al., (1986) Mol. Immunol. 23: 971-982 and Porstmann et al., (1992) J. Immunol. Methods 150: 5-21, incorporated herein by reference in their entireties. Fluorescence counts were read using a FlexStation II fluorescence reader (Molecular Devices LLC, Sunnyvale, Calif.) at an excitation wavelength of 650 nm and an emission wavelength of 690 nm. The K_{D} value was determined by nonlinear regression using GRAPHPAD PRISM.RTM (GraphPad Software, Inc., La Jolla, Calif.).

### Example 3

Western Blotting: Whole cell proteins were harvested from either cell pellets or tumor tissues using T-PER Tissue Protein Extraction Reagent (Pierce Biotechnology, Rockford, Ill.). Total protein (20 μg) was resolved using NuPAGE 4-12% Bis-Tris gels, and immunoblotting was carried out using anti-GPC3 mAb (Clone 1G12, BioMosaics Inc., Burlington, Vt.) at 1:5,000 dilution.

### Example 4

Immunofluorescence and Immunohistochemistry: For immunofluorescence staining of HCC cell lines, cells were seeded onto coverslips 24 h prior to staining. Staining was done using anti-GPC3 mAb (1:500, in PBS with 1% Bovine Serum Albumin (BSA) and 2% normal goat serum) and ALEXA FLOUR.RTM 660 goat-anti-mouse IgG (H+L) (Invitrogen Life Technologies, Carlsbad, Calif.). Staining for GPC3 in xenograft sections was performed using DAKO ENVISION PLUS.RTM Kit (Dako, Carpinteria, Calif., USA).

### Example 5

Bioconjugation and Radiolabeling: Conjugation and radiolabeling of anti-GPC3 mAb (Clone 1G12, BioMosaics Inc., Burlington, Vt.) was performed with zirconium-89 as described by Vosjan et al., (2010) Nat. Protoc. 5: 739-743, incorporated herein by reference in its entirety. In brief, the linker molecule, Df-DFO-NCS (p-isothiocyanatobenzyl-desferrioxamine) was conjugated to anti-GPC3 mAb in Na₂CO₃ buffer (0.1 M. pH 9.0) in a molar ratio of 1:100, followed by purification using SEPHADEX.RTM G-50 spin columns (GE Healthcare, Waukesha, Wis.). Approximately 200 μg of the DFO-conjugated antibodies were then radiolabeled with ⁸⁹Zr by the addition of 37 MBq (1 mCi) of ⁸⁹Zr in 0.1 N sodium acetate buffer (NaOAc, pH 6.0) and incubated for 1 h. The radiolabeled bioconjugates (⁸⁹Zr-DFO anti-GPC3 mAb, abbreviated as ⁸⁹Zr-DFO-GPC3) were then purified using PD-10 columns. The labeling yield was approximately 20%.

### Example 6

Cellular Uptake and Internalization Assays: In vitro cell uptake assays of ⁸⁹Zr-DFO-GPC3 in cell lines were performed as previously in Miao et al., (2010) Bioconjug. Chem. 21: 947-954, incorporated herein in its entirety, with minor modifications. Cells were seeded at a density of 2.0×10⁵ per well onto 12-well plates and allowed to attach overnight. Cells were washed twice with serum-free DMEM medium and incubated with ⁸⁹Zr-DFO-GPC3 (2 μCi per well, 74 kBq, approximately 0.2 μg) in 400 μL of serum-free McCoy 5 medium at 37° C. After 0.5 h, 1 h, 2 h, and 4 h, the cells were washed three times with cold PBS and lysed in 200 μL of 0.2 M NaOH. The radioactivity of the cells was counted using a Perkin Elmer 1470 automatic γ-counter (Perkin Elmer, Waltham, Mass.). The protein concentration of each sample was measured by the bicinchoninic acid (BCA) assay (Pierce Biotechnology, Rockford, Ill.) and cell uptake data were expressed as the percentage of the applied radioactivity per mg protein.

For cell internalization assays, HepG2 cells (5×10⁵ per well) were seeded in 6-well tissue culture plates and allowed to attach overnight. Cells were washed twice with serum-free DMEM medium and incubated with ⁸⁹Zr-DFO-GPC3 (5 μCi per well, 185 kBq, approximately 0.5 μg) in 1 mL of serum-free DMEM medium at 37° C. After 0.5 h, 6 h, 12 h, 24 h, and 40 h, the medium was collected and the cells were washed two times with cold PBS. Internalization of the radiolabeled antibody was determined by washing the cells with acid wash buffer (0.2 M glycine/HCI buffer, pH 2.0) for 5 min at 4° C. to remove the membrane-bound radiocomplex and then measuring the remaining internalized radioactivity. Cells were lysed in 500 μL of 0.2 M NaOH and the collected solution was considered as internalized fraction. The radioactivity of all fractions was counted using a PerkinElmer 1470 automatic γ-counter (PerkinElmer, Waltham, Mass.). Data were expressed as percentage of applied radioactivity.

### Example 7

Animal Models (Subcutaneous and Orthotopic): To generate subcutaneous xenografts, approximately 6 to 10 million of HCC cells were suspended in 100 μL Dulbecco's Phosphate Buffered Saline (DPBS) (Invitrogen Life Technologies, Carlsbad, Calif.) and injected subcutaneously near the left forelimb of adult nude mice. Tumors were allowed to grow to approximately 1.0 cm in largest diameter (3-4 weeks after inoculation) before mice were used for in vivo imaging and biodistribution studies.

To generate the orthotopic HCC tumor model, subcutaneous “seed” xenografts originated from HCC cell lines pre-labeled with tri-fusion reporter genes (bioluminescence, fluorescence and PET) were first generated, and then harvested for surgical implantation (as 1 mm×1 mm pieces) into the liver of adult male nude mice (6-8 weeks old) as described in Sun et al., (2011) Neoplasia 13: 735-747, incorporated herein by reference in its entirety. Implanted tumor growth was monitored by in vivo bioluminescence imaging on a weekly basis, and mice with successful implantations were used for PET/CT scanning.

### Example 8

Small Animal PET, PET/CT, and Image Analysis: Small animal PET of mice bearing subcutaneous tumors (n=4 each for each group) was performed using a micro-PET R4 rodent-model scanner (Siemens Medical Solutions USA, Mountain View, Calif.). Mice were injected intravenously with ⁸⁹Zr-DFO-GPC3 (approximately 0.3 MBq (10 μCi, approximately 1 μg)) via the tail vein under isoflurane anesthesia. Starting 24 h post-injection (p.i), static scans (5 min) were acquired every 24 h until 168 h p.i; mice were anesthetized with 2% isoflurane and placed in the prone position near the center of the field of view of the scanner for image acquisition.

Small-animal PET imaging of orthotopic HCC animal models was performed using the Inveon PET/CT scanner (Siemens Medical Solutions USA, Mountain View, Calif.). The tracer, ⁸⁹Zr-DFO-GPC3 (0.3 MBq, 10 μCi, approximately 1 μg), was injected intravenously via the tail vain. Mice were placed on a custom-built four-mouse holder first for CT image acquisition (632 slices at 206 μm) that was used for photon attenuation correction and image co-registration with PET image data for anatomical information. A static 5 min PET scan was then performed for ⁸⁹Zr activity, and was reconstructed using the Ordered Subsets Expectation Maximization (OSEM) 2D algorithm (159 slices with 0.796 mm resolution). Static scans were performed every 24 h, starting 24 h p.i, till 168 h p.i. Region of interest (ROI) analysis was performed using the Inveon Research Workspace software. The maximum % ID/g upon normalization to injected dose was determined every 24 h.

After the final PET or PET/CT scan, the animals were sacrificed by cervical dislocation under deep anesthesia and dissected. Tumors and organs of interest were excised, weighed, and their radioactivity was measured using a Cobra II auto-γ-counter B5002 (Packard, Virginia Beach, Va.). Results are expressed as percent of injected dose per gram of tissue (% ID/g).

### Example 9

Statistical Analysis: Quantitative data were expressed as mean±standard deviation (SD). Means were compared using one-way ANOVA and the student t-test. P values less than 0.05 were considered statistically significant.

We claim:

1. An immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT.

2. The probe of claim 1, wherein the detectable label is a radionuclide selected from the group consisting of: ⁶⁴Cu, ⁶⁷Cu, ⁸⁹Zr, ¹²⁴I, ⁸⁶Y, ⁹⁰Y, ¹¹¹In, ^{123/131}I, ¹⁷⁷Lu, ¹⁸F, and ^{99m}Tc.

3. The probe of claim 2, wherein the detectable label is detectable by positron emission tomography (PET) and is zirconium⁸⁹ (⁸⁹Zr) or copper⁶⁴ (⁶⁴Cu).

4. The probe of claim 1, wherein the detectable label is attached to the anti-GPC3-specific antibody (mAb), or a target-specific fragment thereof, by a linker.

5. The probe of claim 4, wherein the linker is DFO.

6. A pharmaceutically acceptable composition comprising:
an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT, and further comprising a pharmaceutically acceptable carrier.

7. A method of obtaining an image of a hepatocellular carcinoma in a subject animal or human, the method comprising the steps of:
(a) delivering to a subject animal or human a pharmaceutically acceptable composition comprising an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT;
(b) subjecting the subject animal or human to positron emission tomography;
(c) identifying a detectable signal from the probe in the subject animal or human; and
(d) generating an image of the detectable signal, thereby obtaining an image of a hepatocellular carcinoma in a subject animal or human.

8. The method of claim 7, wherein the detectable label is zirconium⁸⁹ (⁸⁹Zr) or copper⁶⁴ (⁶⁴Cu).

9. The method of claim 7, wherein the detectable PET label is attached to the anti-GPC3-specific antibody (mAb) or the target-specific fragment thereof by a linker.

10. The method of claim 9, wherein the linker is DFO.

11. A method of detecting a cell having glypican-3 (GPC3), or population of said cells, in a biological sample, the method comprising the steps of:
(a) obtaining a biological sample from an animal or human subject;
(b) contacting the biological sample with an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT; and
(c) subjecting the biological sample to positron emission tomography, whereupon a detectable signal from the probe indicates the presence of a cell having glypican-3 (GPC3), or population of said cells, in the biological sample.

12. The method of claim 11, wherein the detectable label is zirconium⁸⁹ (⁸⁹Zr).

13. The method of claim 11, wherein the detectable PET label is attached to the anti-GPC3-specific antibody (mAb) or the target-specific fragment thereof by a linker.

14. The method of claim 13, wherein the linker is DFO.

15. A method of determining if a subject animal or human has a hepatocellular carcinoma expressing glypican-3 (GPC3), the method comprising the steps of:
(a) obtaining a biological sample from an animal or human subject;
(b) contacting the biological sample with an immunoconjugate probe specific for glypican-3 (GPC3), the probe comprising an anti-GPC3-specific antibody (mAb) or a target-specific fragment thereof, and a detectable label attached thereto, wherein the detectable label is detectable by positron emission tomography (PET) or SPECT;
(c) subjecting the biological sample to positron emission tomography; and
(d) identifying a detectable signal from the probe, wherein the detection of the probe indicates the presence of a cell having glypican-3 (GPC3), or population of said cells, in the biological sample, thereby indicating the presence of a hepatocellular carcinoma in the subject animal or human.

16. The method of claim 15, wherein the detectable PET label is zirconium⁸⁹ (⁸⁹Zr).

17. The method of claim 15, wherein the detectable PET label is attached to the anti-GPC3-specific antibody (mAb) or the target-specific fragment thereof by a linker.

18. The method of claim 17, wherein the linker is DFO.
